(19) United States
(12) Patent Application Publication
(10) Pub. No.: US 2020/0323999 A1
(43) Pub. Date: Oct. 15, 2020

(54) SALINE NANODROPLETS FOR RADIO-FREQUENCY-ACOUSTIC MOLECULAR IMAGING

(71) Applicant: The Board of Trustees of the Leland Stanford Junior University, Stanford, CA (US)

(72) Inventors: Yun-Sheng CHEN, Champaign, IL (US); Sanjiv S. GAMBHIR, Portola Valley, CA (US)

(21) Appl. No.: 16/831,899

(22) Filed: Mar. 27, 2020

Related U.S. Application Data
(60) Provisional application No. 62/826,077, filed on Mar. 29, 2019.

(51) Int. Cl. A61K 49/22; A61K 49/00

(52) U.S. Cl. CPC A61K 49/225; A61K 49/0034; A61K 49/221

(57) ABSTRACT

A nanodroplet particle useful as an imaging contrast agent comprises a saline solution that upon irradiation with a radiofrequency energy generate a detectable acoustic signal. The saline solution nanodroplets can be encapsulated by a fluorinated shell that maintains the integrity of the nanodroplets. The saline solution and the fluorinated shell form the nanoparticles when sonicated, for example, in the presence of a non-ionic surfactant to form double-emulsion compositions. The nanoparticles of the disclosure may be advantageously useful as imaging contrast agents suitable for providing a detectable signal when administered to an animal or human subject. The nanoparticles can be conjugated to targeting agents such as biomarker or cell-specific ligands so that the nanoparticles may be concentrated to a desired target such as, but not limited to, a suspected tumor.

### CROSS-REFERENCE TO RELATED APPLICATIONS

This application claims priority to U.S. Provisional Patent Application Ser. No. 62/826,077 entitled “SALINE NANODROPLETS FOR RADIO-FREQUENCY-ACOUSTIC MOLECULAR IMAGING” filed on Mar. 29, 2019, the entirety of which is hereby incorporated by reference.

### STATEMENT REGARDING FEDERALLY SPONSORED RESEARCH

This invention was made with government support under grant number NCI U54 CA199075 awarded by the National Institutes of Health. The government has certain rights in the invention.

### TECHNICAL FIELD

The present disclosure is generally related to saline nanodroplets detectable by radiofrequency generation of acoustic energy. The present disclosure is also generally related to methods of manufacture and using of the nanodroplets.

### BACKGROUND

Radio-frequency-acoustic (RF-acoustic) imaging is an emerging technique that generates ultrasound images by illuminating tissue with non-ionizing electromagnetic pulses in the frequency range of 20 kHz-300 GHz (FIG. 1A). Choosing the right frequency band within the RF range can largely determine the imaging quality (contrast and penetration depth). In general, the tissue penetration of electromagnetic (EM) wave is deeper when the frequency of EM wave is lower. Therefore, RF wave in the UHF band (300 MHz-1 GHz) penetrates tissue much deeper than light or microwave (Yao & Wang (2011) Contrast Media Mol. Imaging 6: 332-345; Da & Liangzhong (2007) Asia Optical Fiber Comm. Optoelectronics Conf. 227-229; Ash et al., (2017) Lasers Med. Sci. 32: 1909-1918). For instance, UHF-RF wave can reach 10 mm deep in tissue when it loses 50% of the energy, whereas microwave and light can only reach 1-4 mm and 0.08-0.1 mm deep under the same conditions, respectively (Laqua et al., (2010) Ann. Int. Conf. IEEE Engineering Med. Biol.: 1437-1440; Kruger et al., (2000) Radiology 216: 279-283). As a result, ultrahigh-frequency-radio-frequency-acoustic (UHF-RF-acoustic) imaging such as UHF-RF-acoustic tomography (FIG. 1B) can potentially image much deeper compared to photoacoustic or microwave-acoustic imaging. In addition to imaging depth, it has been reported that in the UHF-RF range (200-500 MHz), the RF-absorption of cancerous and normal tissue is different (Joines et al., (1980) Int. J. Radiation Oncol. Biol. Phys. 6: 681-687; Joines et al., (1989) Med. Phys. 16: 840-844). Imaging within this spectrum would provide the highest endogenous tissue contrast and depth penetration. While the feasibility of UHF-RF-acoustic imaging has been demonstrated in animals and humans (Omar et al., (2012) Med. Phys. 39: 4460-4466; Kruger et al., (2000) Radiology 216: 279-283), in-vivo UHF-RF-acoustic molecular imaging that requires targeted agents to reveal molecular specificity of the diseases has not been reported.

The first challenge of developing UHF-RF-acoustic molecular imaging comes from the limited choice of contrast agents with high UHF-RF absorption. There are two main mechanisms of UHF-RF absorption. One is dielectric heating where the power dissipates from electric dipole relaxations; the other is Joule heating where power loss comes from the charge carriers' collision with the conductor when they are accelerated by the electromagnetic field (Ogunlade & Beard (2016) Med. Phys. 42: 170-181). It is known that in normal and cancerous tissues, Joule heating is a dominant mechanism that is independent of the excitation frequencies; while dielectric relaxation loss follows Debye relaxation and increases with frequencies in the spectral range of UHF (Omar et al., (2012) Med. Phys. 39: 4460-4466). Ideal choices for UHF-RF-acoustic contrast agents are materials that either have high dielectric loss or conductivity at the frequency of interests (300 MHz-1 GHz). Several nanoparticles such as gold, silicon, carbon nanotube, and iron oxide nanoparticles, have been tested in RF-acoustic imaging. Neither gold nor silicon nanoparticles has been successfully demonstrated for in vivo imaging due to their weak RF-acoustic signals cross the entire RF-spectrum. In addition, while several inorganic nanoparticles, such as carbon nanotubes and iron oxide nanoparticles have been reported to produce RF-acoustic signals in the microwave range, they provide very weak RF-acoustic signals in the UHF range (Tamarov et al., (2017) Phys. Chem. Chem. Phys. 19: 11510-11517; Wen et al., (2017) Theranostics 7: 1976-1989; Byrd et al., (2010) SPIE BIOS, 7564: 12; Ogunlade et al., (2016) Medical Physics 42: 170-181).

### SUMMARY

One aspect of the disclosure encompasses embodiments of a composition comprising a double emulsion nanodroplet that generates a detectable acoustic signal when irradiated by a radio frequency, wherein the nanodroplet can comprise (i) a high ionic salt solution liquid core and (ii) a fluorinated shell encapsulating the high ionic salt solution liquid core and having an outer surface, wherein the fluorinated shell can be both hydrophobic and oleophobic.

In some embodiments of this aspect of the disclosure, the composition can further comprise a non-ionic surfactant.

In some embodiments of this aspect of the disclosure, the salt solution can be an aqueous solution of a salt selected from the group consisting of sodium chloride (NaCl), sodium hydroxide (NaOH), potassium iodide (KI), potassium chloride (KCl), magnesium chloride (MgCl₂), and calcium chloride (CaCl₂).

In some embodiments of this aspect of the disclosure, the salt solution can be a solution of sodium chloride.

In some embodiments of this aspect of the disclosure, the fluorinated shell can comprise a perfluoroalkane.

In some embodiments of this aspect of the disclosure, the perfluoroalkane is selected from the group consisting of: perfluoropentane, perfluorohexane, perfluoro-15-crown-5-ether, and perfluorodecalin.

In some embodiments of this aspect of the disclosure, the composition can further comprise a reactive group disposed at the outer surface of the fluorinated shell.

In some embodiments of this aspect of the disclosure, reactive group can be a reactive thiol group.

In some embodiments of this aspect of the disclosure, the reactive thiol group is 1,2-distearoyl-sn-glycero-3-phosphoethanolamine-N-(polyethylene glycol)-thiol (DSPE-PEG₂₀₀₀-SH).

In some embodiments of this aspect of the disclosure, the composition can further comprise at least one functional moiety conjugated to the reactive thiol group, wherein the functional moiety can be a detectable label, a ligand having selective affinity for a cell or a biomarker of a cell, an immunoglobulin or fragment thereof having selective affinity for a cell or a biomarker of a cell, a protecting molecule that reduces immunogenicity of the nanodroplet, or a pharmaceutically active agent.

In some embodiments of this aspect of the disclosure, the detectable label can be a fluorescent dye.

In some embodiments of this aspect of the disclosure, the at least one functional moiety can be conjugated to the reactive thiol group by a linker.

In some embodiments of this aspect of the disclosure, the linker can be sulfosuccinimidyl 4-(N-maleimidomethyl) cyclohexane-1-carboxylate (Sulfo-SMCC).

In some embodiments of this aspect of the disclosure, the fluorescent dye can be indocyanine green (ICG).

In some embodiments of this aspect of the disclosure, the ligand having selective affinity for a cell or a biomarker of a cell can selectively bind to a cell receptor.

Another aspect of the disclosure encompasses embodiments of a method of generating an acoustic image comprising administering to an animal or human subject a pharmaceutically acceptable composition comprising a nanodroplet according to any one of claims of the disclosure; irradiating the animal or human subject with a radio frequency that generates an acoustic signal from the nanodroplet, detecting the acoustic signal; and generating an image of the animal or human subject showing the location of the nanodroplet in the animal or human subject.

Yet another aspect of the disclosure encompasses embodiments of a method of synthesizing an encapsulated high ionic concentration saline nanodroplet, the method comprising the steps: (a) mixing an activated perfluoroether with poly(propylene glycol)-block-poly(ethylene glycol)-block-poly(propylene glycol)bis(2-aminopropyl ether; (b) synthesizing a first emulsion by sonicating a mixture of the product of step (a), a perfluoroalkane, and an aqueous salt solution to generate a first emulsion; and (c) generating a double emulsion by sonicating a mixture of the product of step (c) and a non-ionic surfactant, thereby forming a population of encapsulated saline nanodroplets.

In some embodiments of this aspect of the disclosure, the non-ionic surfactant can be Pluronic F-68.

In some embodiments of this aspect of the disclosure, the method can further comprise the step of functionalizing the encapsulated saline nanodroplets with a reactive group.

In some embodiments of this aspect of the disclosure, the reactive group can be a reactive thiol group.

In some embodiments of this aspect of the disclosure, the reactive thiol group can be 1,2-distearoyl-sn-glycero-3-phosphoethanolamine-N-(polyethylene glycol)-thiol (DSPE-PEG₂₀₀₀-SH).

In some embodiments of this aspect of the disclosure, the method can further comprise at least one functional moiety conjugated to the reactive thiol group, wherein the functional moiety can be a detectable label, a ligand having selective affinity for a cell or a biomarker of a cell, an immunoglobulin or fragment thereof having selective affinity for a cell or a biomarker of a cell, a protecting molecule that reduces immunogenicity of the nanodroplet, or a pharmaceutically active agent.

In some embodiments of this aspect of the disclosure, the detectable label is a fluorescent dye.

In some embodiments of this aspect of the disclosure, the at least one functional moiety is conjugated to the reactive thiol group by a linker.

In some embodiments of this aspect of the disclosure, the linker can be sulfosuccinimidyl 4-(N-maleimidomethyl) cyclohexane-1-carboxylate (Sulfo-SMCC).

In some embodiments of this aspect of the disclosure, the fluorescent dye can be indocyanine green (ICG).

In some embodiments of this aspect of the disclosure, the ligand having selective affinity for a cell or a biomarker of a cell specifically binds to a cell receptor.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1F illustrate radiofrequency generation of acoustic signals FIG. 1A illustrates the spectral range of electromagnetic waves in the radio frequency range, where ultra-high frequency (UHF) is in 300 GHz to 1 GHz range.

FIG. 1B illustrates a perspective view of the experimental set up for UHF-radio frequency acoustic (UHF-RF-acoustic) imaging of mice. The acoustic transducer was mounted on the side to image the x-y plane. The mouse was mounted vertically along the z axis. A translational stage can move the mouse in the +/−z direction and the rotational stage rotates the mouse in the x-y plane. Two RF antennae emit RF pulses (100 ns) to excite the RF-acoustic signals.

FIG. 1C illustrates a side view (x-z plane) of the experimental setup.

FIG. 1D illustrates a top view (x-y plane) of the experimental set up.

FIG. 1E illustrates a point spread function of the RF-acoustic system; the enlarged view shows the RF-acoustic signal pattern generated with a point source.

FIG. 1F illustrates a one-dimensional point spread function of the RF-acoustic system, the full width at half maximum is 1.2 mm.

FIGS. 2A-2H illustrate the preparation of RF-acoustic contrast agents with engineered saline nanodrops.

FIG. 2A illustrates an experimental setup of tube phantom for in vitro imaging with each tube containing different concentrations of electrolytes.

FIG. 2B illustrates a RF-acoustic images of various types of electrolytes, the tubes contain 10 wt % concentration of NaCl, NaI, KCl, MgCl₂, and CaCl₂, and 4 wt % concentration of NaOH, with a tube diameter of 1 mm.

FIG. 2C illustrates a RF-acoustic signal amplitude as a function of the conductivity of the electrolytes NaCl, NaI, KCl, MgCl₂, CaCl₂, and NaOH. The generated UHF-RF-acoustic signal peak amplitude follows approximately a linear trend with the conductivity (R²=0.92).

FIG. 2D illustrates a schematic illustration of nanodroplet synthesis using the double emulsion approach.

FIG. 2E illustrates cryo-transmission-electron-microscopy (Cryo-TEM) images showing nanodroplets with NaCl liquid core (25 wt %) and perfluorocarbon shell (left) and a control nanodroplet with only perfluorocarbon (right).

FIG. 2F illustrates a measured size distribution of the nanodroplets using dynamic light scattering, showing a median diameter of the nanodroplets of about 250 nm.

FIG. 2G illustrates a stability test of nanodroplets with a median diameter of 250 nm with various shells: Δ: soybean oil (control), □: perfluoropentane, ∘: Perfluorohexane, ⋄: Perfluoro-15-crown-5-ether, and ∇: perfluorodecalin.

FIG. 2H illustrates a stability test of nanodroplets with a perfluoropentane shell with different median diameters (□: 250 nm, [symbol] : 450 nm, and ▪: 800 nm).

FIGS. 3A-3F illustrate in vitro contrast-enhanced RF-acoustic imaging showing deep tissue penetration.

FIG. 3A illustrates RF-acoustic signal amplitude of saline nanodroplets (red color) as a function of initial NaCl concentration added in synthesis, and the conductivity of NaCl solution (blue color) at corresponding concentration. The result shows that the UHF-RF-acoustic signal increases as the saline concentration increases and saturates at 25 wt %.

FIG. 3B illustrates a RF-acoustic signal amplitude as a function of nanodroplet concentration with 25 wt % encapsulated saline RF-acoustic signal amplitude was linearly correlated with the concentration of nanodroplets (R²=0.995). The grey dashed line shows the RF-acoustic signal level of physiological saline (0.9 wt %).

FIG. 3C illustrates RF-acoustic signal amplitude as a function of aging time. Nanodroplets (1.8×10⁹ nanodroplets/mL) containing 25 wt % encapsulated saline were measured over 14 days to test their RF-acoustic signal stability. Error bar is the standard deviation (N=5).

FIG. 3D illustrates RF-acoustic signal amplitude of the tube phantom with diameter of 1 mm. Six tubes were imaged, the first four tubes contain nanodroplets (1×10⁹ nanodroplets/mL), the fifth and sixth tubes contain 20-nm gold nanoparticles (AuNPs, 5×10¹¹ nanoparticles/mL) and 100-nm iron oxide nanoparticles (Fe₃O₄, 5×10¹¹ nanoparticles/mL), both were 500× higher concentration than that of the nanodroplet solution to bring RF-acoustic signal of iron oxide above the noise level. The 1× nanodroplets produce 3.2±0.7 times higher RF-acoustic signals than the 500×Fe₃O₄ nanoparticles (p=0.00016, n=5) and 5.1±0.6 times higher signals than the 500× gold nanoparticles. FIG. 3E illustrates the UHF-RF-acoustic signal amplitude of nanodroplets as a function of imaging depth in bovine tissue (photograph with setup on the left). Six inclusions were imaged, which were respectively located at 0.5 cm, 1.8 cm, 2 cm, 2.8 cm, 3.5 cm, and 5 cm from the tissue surface. Each inclusion had a diameter of 3 mm and was filled with nanodroplets/gelatin mixture. The mixture contained 1:1 volume ratio of nanodroplets (3×109 nanoparticles/mL with 25 wt % saline) and 12% gelatin.

FIG. 3F illustrates the Stanford logo phantom containing nanodroplets (8×109 nanodroplets/mL) demonstrated RF-acoustic imaging can penetrate tissue ˜7 cm. The grey shadow near the axis shows the size and shape of the whole tissue.

FIGS. 4A-4D illustrate that GRPR-targeted nanodroplets show high affinity to specific prostate cancer cells and low toxicity.

FIG. 4A illustrates an extinction spectrum of the nanodroplets showing the optical absorption peak from Indocyanine green (ICG) dye.

FIG. 4B illustrates a Western blot analysis of GRPR expression of common prostate cancer cell lines.

FIG. 4C illustrates a cell viability test using non-targeted nanodroplets, showing low cell toxicity up to 3×10¹¹ nanodroplets/mL.

FIG. 4D illustrates an optical fluorescent imaging of targeted nanodroplets incubated with GRPR+ (PC3) and GRPR− (DU145) cell lines.

FIGS. 5A-5G illustrate in vivo RF-acoustic molecular imaging using targeted nanodrops.

FIG. 5A illustrates in vivo mouse imaging with subcutaneous tumors (PC3) using targeted nanodroplets (group 1, n=5), tumors (DU145) using targeted nanodroplets (group 2, n=5), tumors (PC3) with non-targeted nanodroplets (group 3, n=5), and tumors (DU145, n=5) with non-targeted nanodroplets (group 4).

FIG. 5B illustrates a comparison of RF-acoustic signal amplitude in the tumor region with targeted and non-targeted nanodroplets, showing the strongest signal from the targeted nanodroplets with PC3 tumors (*, p=0.007, n=5).

FIG. 5C illustrates epi-fluorescent imaging of mice in groups 1 to 4, showing the strongest fluorescence signals from PC3 tumor bearing mouse with the GRPR-targeted nanodroplets. The scanning volume is 23 mm (x)×19 mm (y).

FIG. 5D illustrates fluorescence imaging of harvested main organs from one mouse in group 1, showing that the nanodroplets mainly accumulated at the tumor and the spleen.

FIG. 5E illustrates fluorescence imaging of the harvested tumors from 4 mice (one from each group), showing the strongest signal from the mouse in group 1 (GRPR-targeted nanodroplets with PC3 tumors), demonstrating their highest binding specificity and efficiency.

FIG. 5F illustrates collective measurements of the fluorescence signals from all the major organs and tumors from the 4 groups of mice, indicating that the nanodroplets mainly accumulated at the tumor and the spleen, but only GRPR-targeted nanodroplets show highest binding specificity at the tumor site (*, p=0.006, n=5, **, p<=0.0005, n=5).

FIG. 5G illustrates immunohistochemical tissue sections of liver, kidney, and spleen from mice with non-targeted nanodroplet or saline injections (100 μL, 1×10¹¹ nanodroplets/mL) over 2 weeks, stained with hematoxylin and eosin (H&E) and Perls' Prussian Blue, showing that there was no noticeable tissue damage from the nanodroplet injections.

FIG. 6 illustrates fluorescent intensity as a function of the thiol concentration. The open dots represent the different concentrations of the standard thiol sample provided by the thiol quantification kit (white filled circles). The relation between the intensity and the thiol concentration was fitted with a linear regression fitting (red line, R²=0.97, n=6). The nanodroplets (1.5×10¹⁵ nanodroplets/mL, black filled circle) contains 2.7±0.5 μmole of thiols which correspond to (6.5±1.2)×10³ thiols per nanodroplet. The experiment was replicated twice with the similar outcome.

FIGS. 7A-7D illustrate a customized phantom holder for RF-acoustic characterization of nanodrops.

FIG. 7A illustrates a three-dimensional schematic of the customized tube-phantom holder.

FIG. 7B illustrates the dimensions of the customized holder for calibration, the transverse view showing the total width of the holder is 30 mm.

FIG. 7C illustrates the vertical view showing each tube phantom is 1 mm in diameter.

FIG. 7D illustrates a digital image of an embodiment of the assembled tube phantom.

FIG. 8 is a graph illustrating the RF-acoustic amplitude as a function of iron oxide nanoparticle concentration. The open dots represent the RF-acoustic amplitude at each concentration, the error bar is standard deviation of signals (n=5), and the dashed line represents the linear regression fitting of the data (R²=0.997). The experiment was replicated twice with the similar outcome.

FIG. 9 is a bar graph illustrating fluorescent intensities of ICG from 30 cancer cells incubated with nanodroplets. PC3 cells with the GRPR-targeted nanodroplets show 2.5-fold higher ICG fluorescent signals (p=0.002, n=30 cells analyzed in both cases), compared with the non-targeted (PEG) nanodroplets (FIG. 10). The negative control cell line DU145 showed much lower fluorescence signals from both targeted and non-targeted nanodroplets due to the relatively low expression of GRPR. *p=0.002244, **p=0.005906, ***p=0.005239, ****p<0.

FIGS. 10A and 10B illustrate fluorescence intensity of ICG-saline nanodroplets as a function of concentration.

FIG. 10A illustrates epi-fluorescence images of ICG-saline nanodroplets. The concentration of nanodroplet from left to right is (1±0.21)×10¹¹ nanodroplets/mL, (2±0.27)×10¹¹ nanodroplets/mL, (3±0.34)×10¹¹ nanodroplets/mL, (4±0.53)×10¹¹ nanodroplets/mL, (5±0.79)×10¹¹ nanodroplets/mL, respectively.

FIG. 10B illustrates fluorescence intensity of ICG-saline nanodroplets as a function of concentration. The open dots are the averaged sum of fluorescence intensity over the area of a single well in 96 well plate (ROI=0.3 cm¹¹, N=5). The x error bar is the standard deviation of concentration (N=5), the y error bar is the standard deviation of 5 independent measurements at each concentration (N=5). The red dashed line is the linear regression fitting of the intensity data, R²=0.97.

FIG. 11 illustrates a bovine tissue holder for RF-acoustic tomography showing that the bovine tissue is mounted in the vertical direction. The two black tubes are at the location marked as circles (labeled as ‘tissue holding rod’) shown in FIG. 3D.

FIGS. 12A-12D illustrates a customized animal holder for RF-acoustic tomography.

FIG. 12A illustrates a three-dimensional schematic of the customized animal holder.

FIG. 12B illustrates a photograph of the customized animal holder produced with 3D printing.

FIG. 12C illustrates a photograph of one of the mice during experiments mounted on the customized animal holder.

FIG. 12D illustrates a setup for RF-acoustic tomography imaging.

FIGS. 13A-13D illustrate simulated RF-acoustic signal using COMSOL. All simulation domain contains one nanodroplet.

FIG. 13A illustrates an incoming RF pulse is assumed to be Gaussian with a FWHM of 100 ns.

FIG. 13B illustrates a simulated RF-acoustic signal at 1 μm away from the nanodroplet. The nanodroplet is assumed to have a core-diameter of 250 nm, the perfluorocarbon shell had a thickness of 40 nm.

FIG. 13C illustrates a simulated heat profile (2D) at the peak RF-acoustic signal intensity.

FIG. 13D illustrates a simulated heat profile (1D) at the peak RF-acoustic signal intensity, showing the abrupt temperature change at the perfluorocarbon shell (PFC). The initial/ambient temperature as 293.15K.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

Prior to describing the various embodiments, the following definitions are provided and should be used unless otherwise indicated.

### Definitions

The term “double emulsion” as used herein refers to complex systems, also called “emulsions of emulsions”, in which the droplets of the dispersed phase contain one or more types of smaller dispersed droplets themselves. Double emulsions can encapsulate hydrophobic and hydrophilic compounds. Techniques based on double emulsions are commonly used for the encapsulation of hydrophilic molecules, which suffer from low encapsulation efficiency because of rapid drug partitioning into the external aqueous phase when using single emulsions. The main issue when using double emulsions is their production in a well-controlled manner, with homogeneous droplet size by optimizing different process variables.

A double emulsion is simply defined as an emulsion in an emulsion. An emulsion is a dispersed, multiphase system consisting of at least two immiscible liquids. The liquid that forms droplets is called the “dispersed phase”, while the liquid in the bulk surrounding the droplets is called the “continuous phase”. The dispersion is usually not stable and phase separation will eventually occur at long time scale.

The double emulsion is a system, in which two liquids are separated by a third liquid which is not miscible with the first two liquids. In the case of water and oil, there are two possible cases of double emulsions: water-in-oil-in-water (w/o/w) emulsion and oil-in-water-in-oil (o/w/o) emulsion. In the former case, each dispersed droplet of water forms a vesicular structure with single or multiple aqueous compartments separated from the continuous aqueous phase by a layer of oil phase.

In two-step emulsification, primary w/o emulsions are formed under high shear conditions, typically from ultrasonification or homogenization. The secondary emulsification is normally carried out without the shear, which would disrupt the already-formed primary emulsions. The composition of the double emulsion is crucial, since with different surfactants, the properties and concentration of the oil phase affect the stability of these emulsions. It is known that the inner hydrophobic emulsifiers must be used in excess (about 10-30% by weight) while the hydrophilic emulsifiers are used in low concentration (0.5-5% by weight) in order to maintain stability.

The terms “subject”, “individual”, or “patient” as used herein are used interchangeably and refer to an animal preferably a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. In a particular embodiment, the mammal is a human. In other embodiments, animals can be treated; the animals can be vertebrates, including both birds and mammals. In aspects of the disclosure, the terms include domestic animals bred for food or as pets, including equines, bovines, sheep, poultry, fish, porcines, canines, felines, and zoo animals, goats, apes (e.g. gorilla or chimpanzee), and rodents such as rats and mice.

In the context of certain aspects of the disclosure, the term “subject” generally refers to an individual who will receive or who has received treatment (e.g., administration of a compound of the disclosure, and optionally one or more other agents) for a condition characterized by a cancer. In certain aspects, a subject may be a healthy subject. Typical subjects for treatment include persons afflicted with or suspected of having or being pre-disposed to a disease disclosed herein, or persons susceptible to, suffering from or that have suffered a disease disclosed herein. A subject may or may not have a genetic predisposition for a disease disclosed herein.

The terms “administering” and “administration” as used herein refer to a process by which an amount of a compound of the disclosure or compositions contemplated herein are delivered to a subject for prevention and/or treatment purposes. Compositions are administered in accordance with good medical practices taking into account the subject's clinical condition, the site and method of administration, dosage, patient age, sex, body weight, and other factors known to physicians.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

The term “pharmaceutically acceptable carrier, excipient, or vehicle” as used herein refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “thio” as used herein refers to a compound described previously herein wherein a carbon or oxygen atom is replaced by a sulfur atom.

The term “biomarker” as used herein refers to an antigen such as, but not limited to, a peptide, polypeptide, protein (monomeric or multimeric) that may be found on the surface of a cell, an intracellular component of a cell, or a component or constituent of a biofluid such as a soluble protein in a serum sample and which is a characteristic that is objectively measured and evaluated as an indicator of a tumor or tumor cell. The presence of such a biomarker in a biofluid or a biosample isolated from a subject human or animal can indicate that the subject is a bearer of a cancer. A change in the expression of such a biomarker may correlate with an increased risk of disease or progression, or predictive of a response of a disease to a given treatment. Exemplary biomarkers useful in the systems and methods of the disclosure can be, but are not limited to, such as activin A; IL-18 BPa, adiponectin/acrp30, IL-18 receptor α/IL-1 R5, AgRP, IL-18 receptor β/AcPL, ALCAM, IL-2 receptor α, angiogenin, IL-2 receptor α, AR (amphiregulin), IL-3, Axl, IL-4, B7-1/CD80, I-TAC/CXCL11, BCMA/TNFRSF17, leptin (OB), BDNF, LIF, β-NGF, LIGHT/TNFSF14, BLC/BCA-1/CXCL13, LIGHT/TNFSF14, BMP-5, MCP-2, BTC, MCP-3, cardiotrophin-1/CT-1, MCP-4/CCL13, CTLA-4/CD152, M-CSF, CXCL16, MMP-10, Dtk, MMP-13, EGF, MMP-9, EGF receptor/ErbB1, MSP α-chain, endoglin/CD105, MSP β-chain, eotaxin/CCL11, NAP-2, eotaxin-2/MPIF-2, NGF R, eotaxin-3/CCL26, NT-4, ErbB3, OSM, Fas/TNFRSF6, osteoprotegerin, Fas Ligand, PDGF receptor β, FGF Basic, PDGF-AA, FGF-4, PDGF-AB, FGF-6, PDGF-BB, FGF-7/KGF, PIGF, FGF-9, P-selectin, follistatin, RAGE, GITR/TNFRF18, RANTES, HB-EGF, SCF, HCC-4/CCL16, SCF receptor/CD117, HGF, sgp130, 1-309, Siglec-9, IGFBP-1, siglec-5/CD170, IGFBP-2, Tarc, IGFBP-3, TGFα, IGF-I, TNF RI/TNFRSF1A, IGF-I, TNF RII/TNFRSF1B, IGF-I S receptor, TNFβ, IGF-II, TRAIL R1/DR4/TNFRSF 10/, IGF-II, TRAIL R3/TNFRSF 10C, IL-1α, TRAIL R4/TNFRSF 10D, IL-1β, TRANCE, IL-1 R4/ST2, TREM-1, IL-1 sRI, TROP/TNFRSF19, IL-1 sRI, uPAR, IL-10, VCAM-1 (CD106), IL-10 receptor β, VE-cadherin, IL-13 receptor α1, VEGF, IL-13 receptor α2, VEGF R2 (KDR), IL-17, VEGF R3, and the like, or any combination thereof. It is considered within the scope of the disclosure for a cancer or cancer cell to be characterized by at least one biomarker and more typically by a plurality (a panel) of such markers.

The term “cancer”, as used herein, shall be given its ordinary meaning, as a general term for diseases in which abnormal cells divide without control. In particular, cancer refers to angiogenesis related cancer. Cancer cells can invade nearby tissues and can spread through the bloodstream and lymphatic system to other parts of the body.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, bladder cancer, breast cancer, colorectal cancer, endometrial cancer, head and neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease leukemia, acute lymphoblastic leukemia, acute myeloid leukemia, liver cancer, medulloblastoma, neuroblastoma, brain tumors generally, non-Hodgkin's lymphoma, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others.

The term “antibody” as used herein, refers to polyclonal and monoclonal antibody preparations, as well as preparations including hybrid antibodies, altered antibodies, F(ab′)₂ fragments, F(ab) fragments, Fv fragments, single domain antibodies, chimeric antibodies, humanized antibodies, and functional fragments thereof which exhibit immunological binding properties of the parent antibody molecule.

The term “detectable moiety” as used herein refers to various labeling moieties known in the art. Said moiety may be, for example, a radiolabel (e.g., ³H, ¹²⁵I, ³⁵S, ¹⁴C, ³²P, etc.), detectable enzyme (e.g., horse radish peroxidase (HRP), alkaline phosphatase etc.), a dye, a colorimetric label such as colloidal gold or colored glass or plastic (e.g., polystyrene, polypropylene, latex, etc.), beads, or any other moiety capable of generating a detectable signal such as a colorimetric, fluorescent, chemiluminescent or electrochemiluminescent (ECL) signal.

The term “dye” as used herein refers to any reporter group whose presence can be detected by its light absorbing or light emitting properties. For example, Cy5 is a reactive water-soluble fluorescent dye of the cyanine dye family. Cy5 is fluorescent in the red region (about 650 to about 670 nm). It may be synthesized with reactive groups on either one or both of the nitrogen side chains so that they can be chemically linked to either nucleic acids or protein molecules. Labeling is done for visualization and quantification purposes. Cy5 is excited maximally at about 649 nm and emits maximally at about 670 nm, in the far red part of the spectrum; quantum yield is 0.28. FW=792. Suitable fluorophores(chromes) for the probes of the disclosure may be selected from, but not intended to be limited to, fluorescein isothiocyanate (FITC, green), cyanine dyes Cy2, Cy3, Cy3.5, Cy5, Cy5.5 Cy7, Cy7.5 (ranging from green to near-infrared), Texas Red, and the like. Derivatives of these dyes for use in the embodiments of the disclosure may be, but are not limited to, Cy dyes (Amersham Bioscience), Alexa Fluors (Molecular Probes Inc.), HILYTE™ Fluors (AnaSpec), and DYLITE™ Fluors (Pierce, Inc).

The term “fluorescence” as used herein refers to a luminescence that is mostly found as an optical phenomenon in cold bodies, in which the molecular absorption of a photon triggers the emission of a photon with a longer (less energetic) wavelength. The energy difference between the absorbed and emitted photons ends up as molecular rotations, vibrations or heat. Sometimes the absorbed photon is in the ultraviolet range, and the emitted light is in the visible range, but this depends on the absorbance curve and Stokes shift of the particular fluorophore.

The term “nanoparticle” as used herein refers to a particle having a diameter of between about 1 and about 1000 nm, preferably between about 100 nm and 1000 nm, and most preferably between about 50 nm and 700 nm. Similarly, by the term “nanoparticles” is meant a plurality of particles having an average diameter of between about 50 and about 1000 nm.

The detectable signal is defined as an amount sufficient to yield an acceptable image using equipment that is available for pre-clinical use. A detectable signal maybe generated by one or more administrations of the probes of the present disclosure. The amount administered can vary according to factors such as the degree of susceptibility of the individual, the age, sex, and weight of the individual, idiosyncratic responses of the individual, the dosimetry, and the like. The amount administered can also vary according to instrument and digital processing related factors.

The term “in vivo imaging” as used herein refers to methods or processes in which the structural, functional, or physiological state of a living being is examinable without the need for a life-ending sacrifice.

The term “non-invasive in vivo imaging” as used herein refers to methods or processes in which the structural, functional, or physiological state of a being is examinable by remote physical probing without the need for breaching the physical integrity of the outer (skin) or inner (accessible orifices) surfaces of the body.

The term “perfluoroalkane” (PFC) as used herein refers to organofluorine compounds with the formula CₓF_{y}, i.e. they may contain only carbon and fluorine. Compounds with the prefix “perfluoro-” are hydrocarbons, including those with heteroatoms, wherein all C—H bonds have been replaced by C—F bonds. Perfluoroalkanes are very stable because of the strength of the carbon-fluorine bond, the strength of which is a result of the electronegativity of fluorine imparting partial ionic character through partial charges on the carbon and fluorine atoms, which shorten and strengthen the bond through favorable covalent interactions. Multiple carbon-fluorine bonds increase the strength and stability of other nearby carbon-fluorine bonds on the same geminal carbon. Furthermore, multiple carbon-fluorine bonds strengthen the “skeletal” carbon-carbon bonds from an inductive effect. Saturated fluorocarbons are more chemically and thermally stable than their corresponding hydrocarbon counterparts, and indeed any other organic compound. Fluorocarbons are colorless and have high density, up to over twice that of water. They are not miscible with most organic solvents (e.g., ethanol, acetone, ethyl acetate, and chloroform), but are miscible with some hydrocarbons (e.g., hexane in some cases). They have very low solubility in water, and water has a very low solubility in them (on the order of 10 ppm). They have low refractive indices. Fluorocarbons have low intermolecular attractive forces and are lipophobic in addition to being hydrophobic and non-polar. They exhibit low viscosities when compared to liquids of similar boiling points, low surface tension and low heats of vaporization. Most fluoroalkanes are liquids; the most notable exception is perfluorocyclohexane, which sublimes at 51° C. Fluorocarbons also have low surface energies and high dielectric strengths.

### DISCUSSION

Ultra-high-frequency-radio-frequency-acoustic (UHF-RF-acoustic) imaging is a translational imaging modality that can image living subjects with millimeters to sub-millimeter spatial resolution and centimeters of imaging depth, advantageous compared to photoacoustic and optical imaging techniques for deep-seated targets. However, current contrast agents in other RF range produce very weak signal in the UHF range. While biocompatible electrolytes such as hypertonic saline can produce strong UHF-RF-acoustic signals, they cannot simply be conjugated with molecular targeting moieties. Prior attempts to encapsulate highly concentrated electrolytes into conjugatable nanoparticles have failed due to the high osmotic pressure. Thus, UHF-RF-acoustic has not been used for targeting molecular imaging of any disease. A new approach was developed to produce nanodroplets that can stably encapsulate hypertonic saline. These saline nanodroplets are designed to generate significantly strong RF-acoustic signals that are about 1500-times higher than the concentration-matched commercially available iron-oxide nanoparticles, a widely used RF-acoustic (in microwave) and MRI medical imaging agents. Finally, the first in-vivo UHF-RF-acoustic molecular imaging of gastrin releasing peptide receptor, a biomarker associated with prostate, breast, colon, and lung cancers progression was demonstrated using the targeted nanodroplets in a prostate cancer xenograft mouse model.

The disclosure accordingly provides embodiments of nanodroplet particles comprising a saline solution that upon irradiation with a radiofrequency energy generate a detectable acoustic signal. The saline solution nanodroplets can be encapsulated by a fluorinated shell that maintains the integrity of the nanodroplets. The saline solution and the fluorinated shell form the nanoparticles when sonicated, for example, in the presence of a non-ionic surfactant to form double-emulsion compositions. The nanoparticles of the disclosure may be advantageously used as imaging agents suitable for providing a detectable signal when administered to an animal or human subject. By derivatizing the nanoparticles by conjugating targeting agents such as biomarker or cell-specific ligands the nanoparticles may be concentrated to a desired target such as, but not limited to, a suspected tumor. The conjugation of a detectable label such as a dye further allows additional means of detecting the presence of the nanoparticles in the animal or human subject. Further provided are methods to obtain an image of the localization of the nanoparticles within the subject and the methods of synthesizing the nanoparticles.

Aiming to pinpoint cancers at early stages, molecular imaging is a technique designed to reveal both the anatomical information and cancer-associated molecular events simultaneously (Weissleder R. (2006) Science 312: 1168-1171; Gambhir, S. S. (2002) Nat. Revs. Cancer 2: 683). While molecular imaging promises to transform cancer diagnosis, the need for early detection has not yet been met by existing technology and research. To detect cancer at the molecular level simultaneously requires high contrast, high sensitivity, deep imaging depth, as well as high spatial resolution. However, enhancing one capability often leads to sacrificing another, therefore no comprehensive molecular imaging technique is currently available for diagnosing early stage deep-seated tumors and their precursors clinically.

Positron emission tomography (PET) and single-photon emission computed tomography (SPECT) offer high sensitivity but provide moderate imaging resolution; computed tomography (CT) and magnetic resonance imaging (MRI) provide millimeter spatial resolution with relatively low molecular sensitivity; molecular ultrasound imaging provides high spatial resolution down to micrometers but suffers from the non-extravasation of contrast agents (Willmann et al., (2008) Nat. Revs. Drug Disc. 7: 591; Hussain & Nguyen (2014) Adv. Drug Deliv. Revs. 66: 90-100; Omar et al., (2012) Med. Phys. 39: 4460-4466). As such, developing new molecular imaging techniques that can overcome these limitations remains an outstanding challenge for early cancer diagnosis. Ultra-high-frequency-radio-frequency-acoustic (UHF-RF-acoustic) imaging has been demonstrated to generate deep-tissue high-contrast images with resolution comparable to clinical ultrasonography and can potentially extend to whole-body imaging (Kruger et al., (1999) Med. Phys. 26: 1832-1837; Eckhart et al., (2011) IEEE Trans. Biomed. Engin. 58: 2238-2246; Ye et al., (2016) IEEE Trans. Med. Imaging 35: 839-844; Patch & See (2015) IEEE Great Lakes Biomed. Conf. (GLBC) 1-4). However, past attempts of developing suitable molecular agents have failed because of trade-offs between biocompatibility and signal enhancement. The present disclosure provides an UHF-RF-acoustic contrast agent using engineered saline nanodroplets. The nanodroplet contrast agents of the disclosure were conjugated with molecular moieties and gastrin-releasing peptide receptor (GRPR)-targeting ligands and demonstrate UHF-RF-acoustic molecular imaging using such contrast agents that could identify, for example, prostate cancer in vivo.

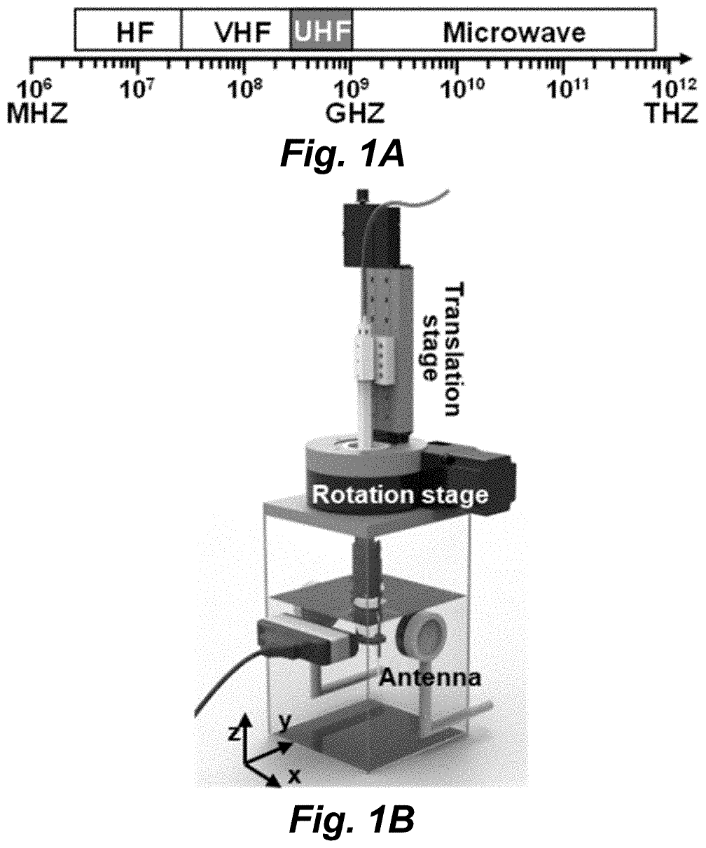

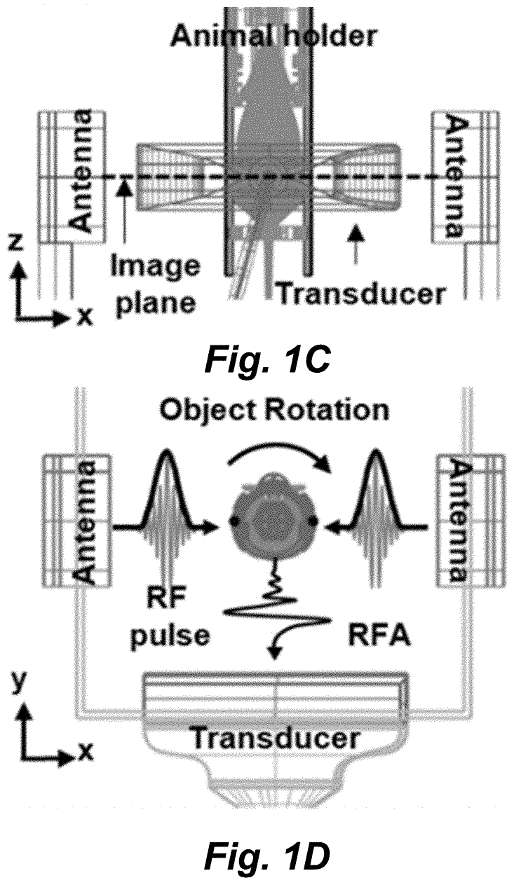

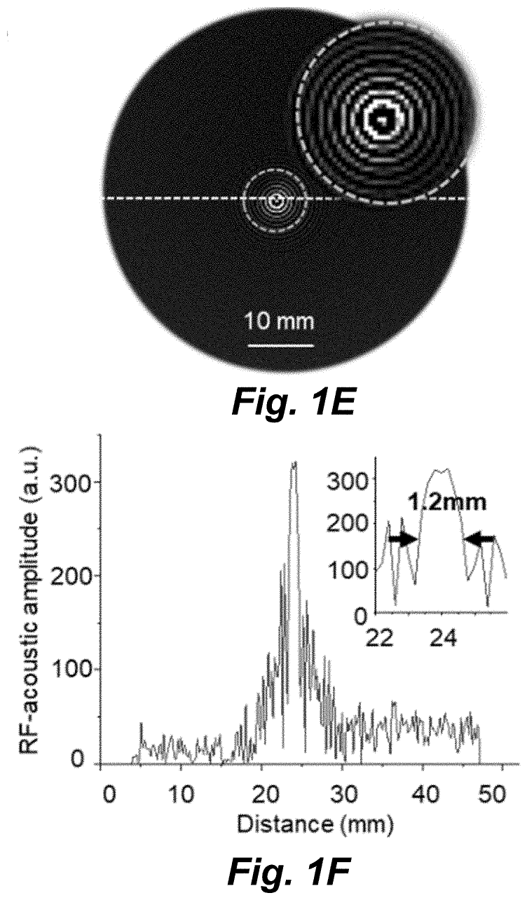

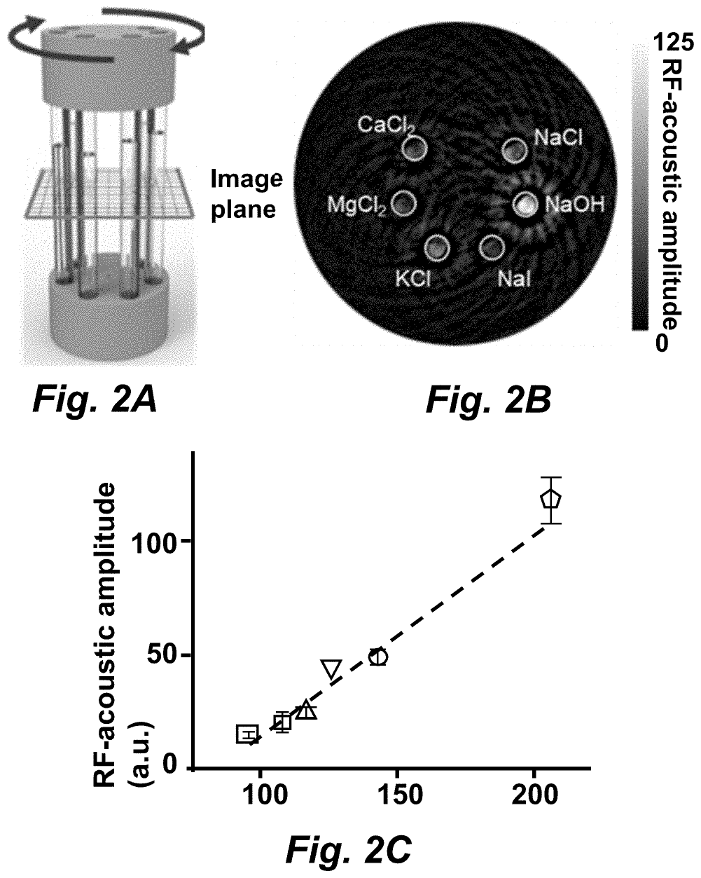

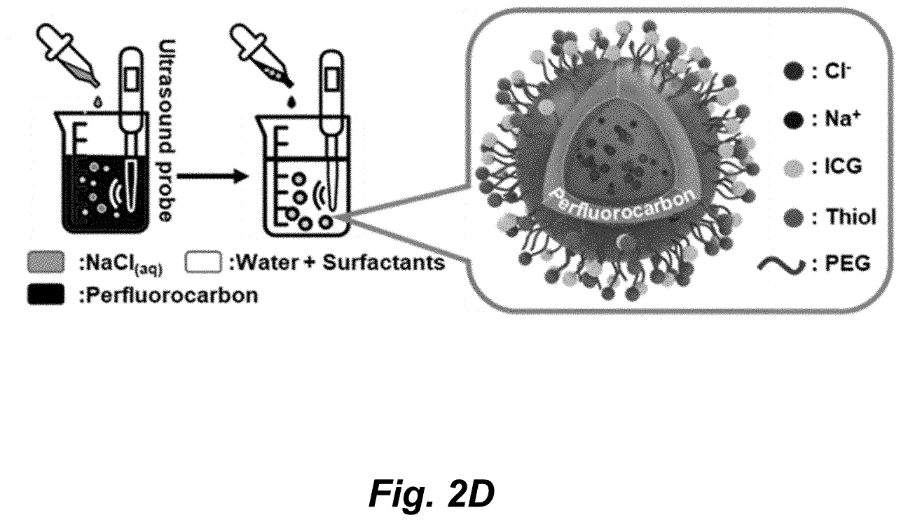

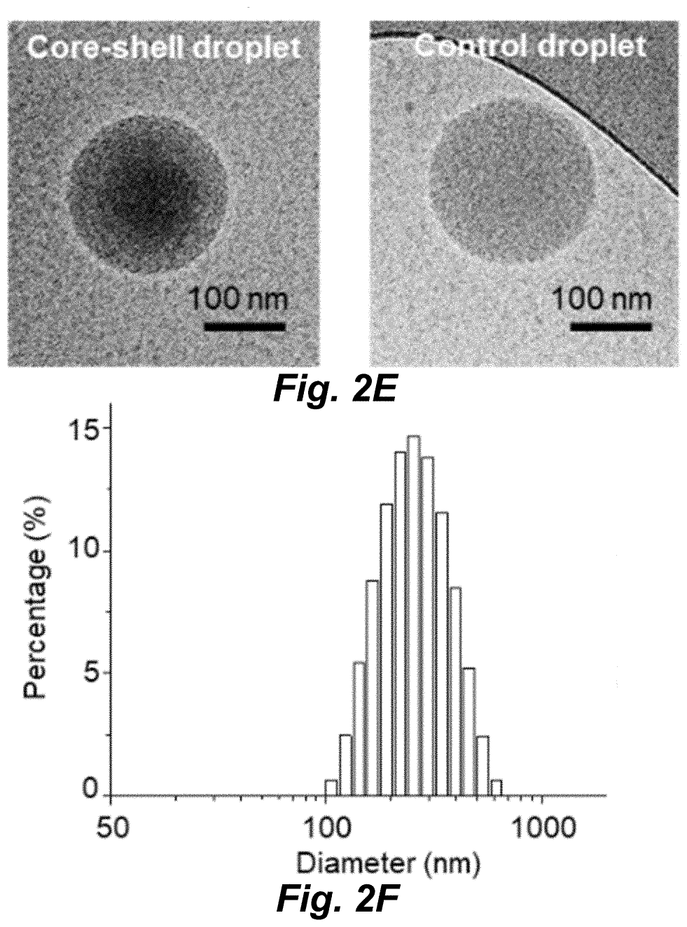

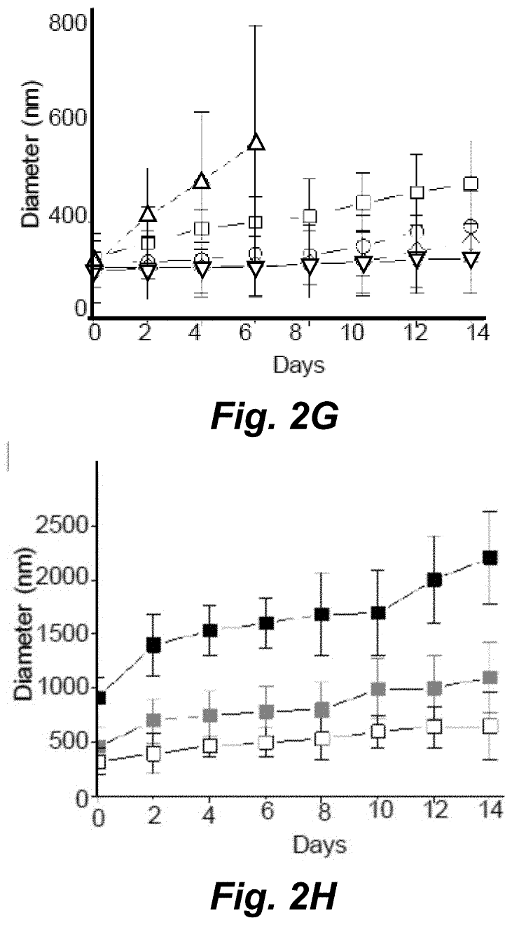

Liquid electrolytes with high ionic conductivity such as aqueous salt solutions produce much stronger RF-acoustic signals than solid-state nanoparticles, thus they may be a better choice for developing UHF-RF-acoustic contrast agents. To determine the optimal composition of salt solutions for UHF-RF-acoustic contrast agents, five different salts were chosen, NaCl_{(aq)}, KI_{(aq)}, KCl_{(aq)}, MgCl_{2(aq)}, and CaCl_{2(aq)} based on their ionic conductivity, solubility in water, and biocompatibility. NaOH_{(aq)} was selected as a control because of its high ionic conductivity but low biocompatibility. These salts were prepared at the same concentration (10 wt %) and separately prepared NaOH_{(aq)} at 4 wt % such that its UHF-RF signals fall in a comparable range of the others. Their UHF-RF-acoustic signals were measured using a prototype RF-acoustic tomography system with a nanosecond UHF-RF pulse at 433 MHz since this frequency is within the medical device radio-communications service band (MedRadio) (FIGS. 1B-1D). This system allows for rotating the samples 360 degrees to generate tomographic images. The generated RF-acoustic signals were collected by an ultrasound-linear-array transducer with a central frequency at 3.5 MHz that provides 1.2 mm imaging resolution on the transverse plane (FIGS. 1E-1F). Customized 3D printed holders were used for phantom imaging (FIG. 2A) and later in vivo mouse imaging (FIGS. 1B-1D).

FIG. 2B shows that NaOH_{(aq)} produces the strongest RF-acoustic signal, followed by KCl_{(aq)} and NaCl_{(aq)}. Plotting the RF-acoustic amplitude of these electrolyte solutions as a function of their conductivity showed that the RF-acoustic amplitude is linearly proportional to their conductivity (R²=0.92), confirming that RF-acoustic signal is mainly related to the conductivity, but minimally associated with the types of salts (FIG. 2C).

An optimal constituent material for UHF-RF-acoustic contrast agents is the salt solution with the highest conductivity. Although NaOH_{(aq)} is highly conductive, it is highly bio-incompatible. While NaCl (saline) produces weaker signals than KCl at 10 wt % in FIG. 2C, it has the highest electrical conductivity among these salts at its saturated concentration (approximately 25 wt %). From the imaging prospective, saturated hypertonic saline should produce the highest RF-acoustic signals and could be a good constituent material for developing RF-acoustic contrast agents.

Salts in water are ions that usually diffuse away in tissue and do not accumulate specifically in diseased sites. Ions also cannot be directly conjugated to molecular targeted ligands, such as antibodies and peptides. To synthesize a RF-acoustic molecular imaging agent, saline needs to be first encapsulated into a stable nanoparticle form for subsequent conjugation with molecular targeted ligands. Many approaches using hydrophobic biomaterials (such as lipid and biodegradable polymers) to encapsulate aqueous droplets to create emulsions (nanodroplets in solution) have been widely used in pharmaceutical and medical diagnostics, in cosmetics and food industry, and in imaging and optics. Unfortunately, none of these existing approaches can encapsulate hypertonic saline to produce stable nanoparticles because of the outward osmotic pressure. This pressure is induced by the large differential ionic concentrations when hypertonic saline is encapsulated as the core of the nanodroplets, and can cause rapid swelling, bursting, and leakage of NaCl from nanoparticles.

Classical osmotic pressure model, described by the van't Hoff law, is defined as the force caused by collision of solute “particles” against a “semipermeable” membrane. The osmotic pressure is related to temperature, concentration difference of solute inside and outside the membrane, and permeability of the solute and solvent through the membrane. Previous theoretical studies have shown that altering solute and water permeability of the oil phase will greatly affect the size of the nanodroplets, suggesting that reducing water permeability can significantly reduce the swelling. Macroscopically, water permeability is directly related to bulk water solubility of the material. While most edible oil and fats have a low water solubility (approximately 0.075%-0.25%), perfluorocarbon liquids, on the other hand, have much lower water solubility by one to two orders of magnitude (e.g., 2.2×10⁻³% in perfluoroheptane and 2.0×10⁻³% in perfluorohexane). To test how water solubility affects stability of nanodroplets, several perfluorocarbon liquids that have been widely used in biomedical applications for ultrasound contrast agents and blood substitutes, including perfluoropentane, perfluorohexane, perfluoro-15-crown-5-ether, and perfluorodecalin as the “oil phase”, and a vegetable oil, a commonly used hydrophobic hydrocarbon liquid for emulsion, as the “reference” oil phase since it has relatively higher solubility of water than perfluorocarbon liquid were chosen.

To stably encapsulate hypertonic saline (up to 25 wt % of NaCl) in a nanoparticle form, a double emulsion approach was developed (FIG. 2D). A customized fluoro-surfactant was used to stabilize the inversed saline-to-perfluorocarbon emulsion and used FDA-approved Pluronic F68 to disperse the first emulsion to form a double emulsion. The first emulsion (oil phase droplets in aqueous solution) and the second emulsion (core-shell saline droplets in aqueous solution) were produced with the assistance of ultrasound agitation and subsequent extrusion for size selection. The functional groups, thiol groups, were then added by post-addition method using 1,2-distearoyl-sn-glycero-3-phosphoethanolamine-N-(polyethylene glycol)-thiol (DSPE-PEG₂₀₀₀-SH) for later conjugating with molecular targeted ligands and indocyanine green (ICG) dye for both molecular targeting and visualization (FIG. 2D).

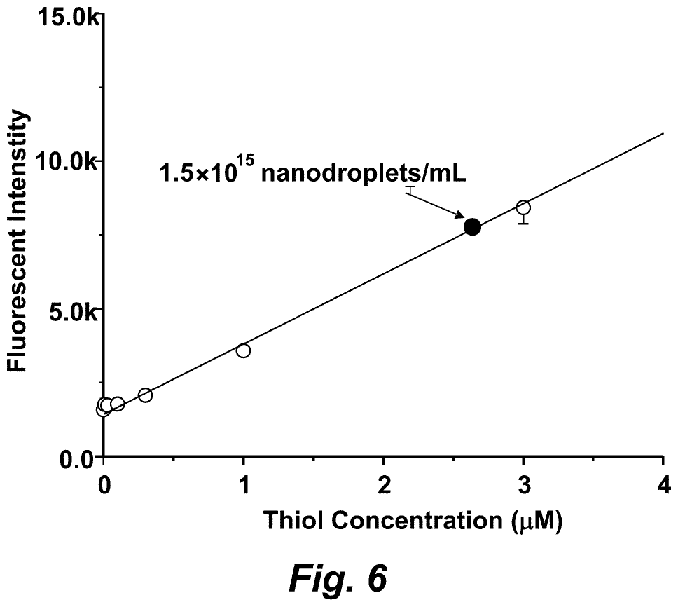

The average numbers of thiol groups on the surface of each nanodroplets was quantified using thiol fluorometric assay. The result indicates that every nanodroplet contains (6.5±1.2)×10³ thiols in average (FIG. 6). To confirm that saline has been encapsulated, cryo-transmission-electron-microscopy (cryo-TEM) was used to image the nanodroplets (double-emulsion). For comparison, the single-phase perfluorohexane droplets were also prepared. The cryo-TEM images (FIG. 2E) show the differential contrasts inside the double-emulsion confirming the core-shell configuration (left panel), whereas a uniform contrast of the single-phase droplets as expected (right panel). Further, the size of nanodroplets was controlled by adjusting the ultrasound power, the composition of the formatting solution, and the pore size of extrusion filter, resulting in a polydispersity of sizes less than 0.1 (FIG. 2F).

To test if this approach results in improved stability, the size changes of the nanodroplets were monitored over two weeks using dynamic light scattering (DLS). FIG. 2G shows that the vegetable-oil nanodroplets increased in size by about 215% after only 6 days, whereas the perfluorocarbon nanodroplets maintain their sizes much better over the same period (e.g., the perfluorodecalin nanodroplets grew only about 18%). After 6 days, due to the large polydispersity of vegetable-oil nanodroplet could no longer be measured by their size using DLS, while the size of the other perfluorocarbon nanodroplets were measured until day 14. Interestingly, the increases in diameters for nanodroplets with perfluorodecalin, perfluoro-15-crown-5-ether, perfluorohexane, and perfluoropentane were 18±15%, 60±21%, 65±17%, and 103±18% after 14 days, respectively (p=0.2273, p=0.0007, p<0.0001, p<0.0001, relative to day 0). It shows the types of perfluorocarbon liquid also affect the stability of nanodroplets.

Different types of single-emulsions (perfluorocarbon liquid-in-water nanodroplets) affecting the stability of emulsions have been reported. In the single emulsion, molecular diffusivity is one critical factor that affects its stability. Typically, decrease in diffusivity results in the decrease of the water solubility, increase of interfacial tension, and slows down the rate of Ostwald ripening, a process that molecules diffuse from small droplets to large droplets, and thus, increases stability of the emulsions. Similar effects may be applied to the double emulsion nanodroplets except the different molecular diffusivities of perfluorocarbons could also lead to the different rates of inward water permeability. Yet, the molecular diffusivities of the perfluorocarbon molecules in nano-emulsions are difficult to measure. On the other hand, a strong relation between the molecular diffusivity and the vapor pressure has been experimentally demonstrated. In a hydrocarbon emulsion, the “oil phase” with a higher vapor pressure has a higher molecular diffusivity, which leads to a low interfacial tension because of the weak interactions between molecules.

It is possible that the relationship between vapor pressure and molecular diffusivity of perfluorocarbon in the nanodroplets is similar to that in the hydrocarbon emulsions, and hypothesized that vapor pressure can indirectly affect the stability of nanodroplets. To examine how vapor pressure affects the stability, the vapor pressure of perfluorocarbon was changed within the same type of perfluorocarbon nanodroplets. When the surfactants and surrounding solvent are the same, it is known that the vapor pressure of perfluorocarbon liquid in the emulsion is mainly affected by the size-dependent Laplace pressure, a work balance between tension and applied pressure at the curved interface of particles. When the size of nanodroplets decreases, Laplace pressure increases and leads to a high vapor pressure. Similar effects have been experimentally demonstrated in the phase-changing-perfluorocarbon single phase emulsions, perfluorocarbon nanodroplets that can be vaporized from liquid nanodroplets to gas microbubbles by ultrasound exposure.

It has been shown that small nanodroplets require a higher energy to be vaporized because the Laplace pressure increases vapor pressure causing the increase of boiling point of the perfluorocarbon in the nanodroplets. In the experiment, the average sizes of the nanodroplets was increased to about 450 nm and about 800 nm in diameter respectively, aiming to decrease the Laplace pressure and consequently reduce the vapor pressure of perfluorocarbon. Perfluoropentane was used to test the hypothesis because their boiling points are close to room temperature at 28° C., and are at the boundary of becoming unstable and more prone to any size change. FIG. 2H confirms that among the perfluoropentane nanodroplets, when the diameter of nanodroplets increased from 250-nm to 800-nm, the increase of nanodroplets for 250-nm, 450-nm, and 800-nm after 14 days were 103±18%, 139±18%, and 142±20%, respectively. The result indicates increase size of nanodroplets indeed reduce its stability. Overall, the results suggest that using high vapor pressure of perfluorocarbon liquids would improve the stability of saline nanodroplets.

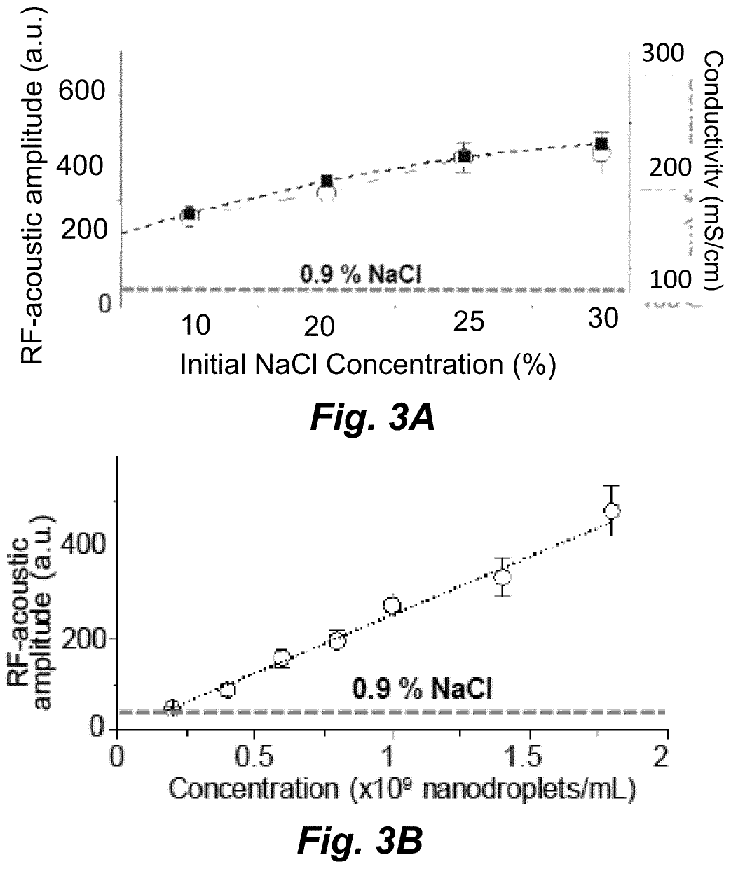

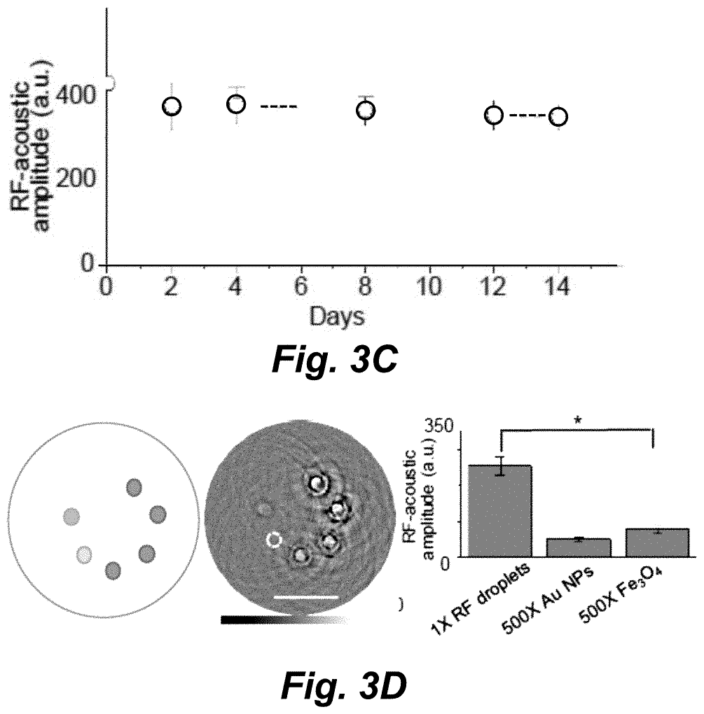

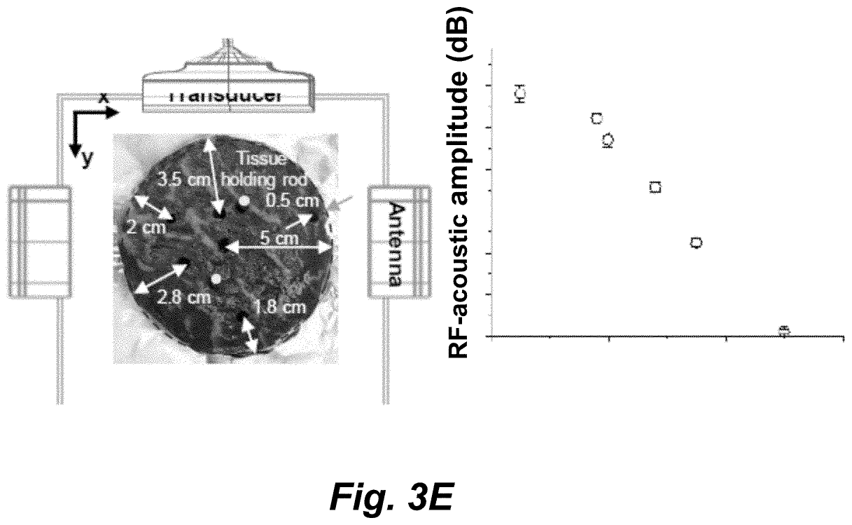

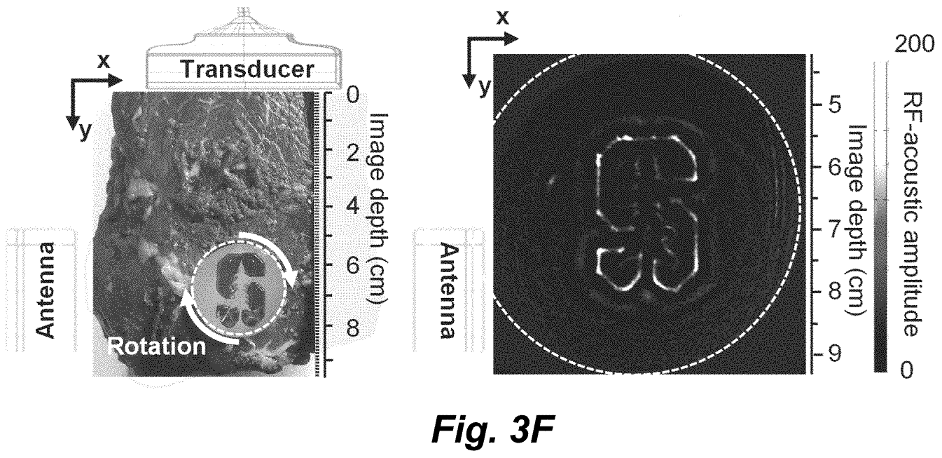

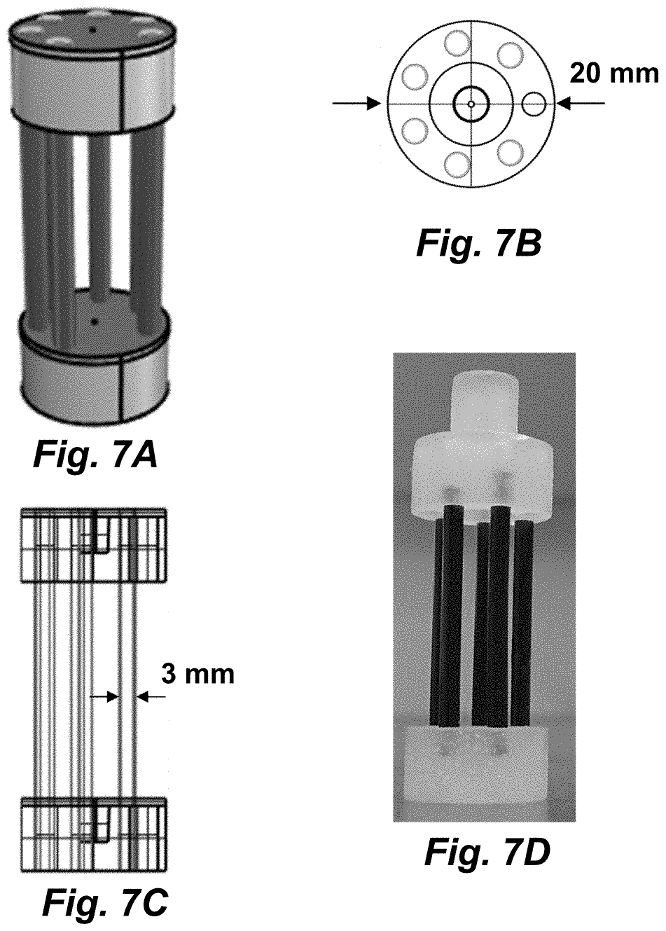

Characterization of the RF-acoustic signals from these nanodroplets was in two steps: first in a tube phantom (FIGS. 3A-3D and 7), and second in an animal tissue phantom (FIGS. 3E and 3F). In the tube phantom study, 4 initial concentrations of NaCl_{(aq)} solutions (25 wt %, 20 wt %, 15 wt %, and 10 wt %) were selected to produce the saline nanodroplets and the tubes containing saline nanodroplet solutions (2×10⁹ nanodroplets/mL). For comparison, a tube was included with only physiological saline (0.9 wt % of NaCl) to mimic RF-acoustic background of tissue. The RF-acoustic signals of the nanodroplets increased linearly with the conductivity of each initial saline concentration (R²=0.99), which further confirmed the encapsulation of saline within these nanodroplets (FIG. 3A). RF-acoustic signals of the nanodroplets (with 25 wt % saline) were recorded the as a function of the nanodroplet concentrations (FIG. 3B). RF-acoustic signal linearly correlate with the concentration of the nanodroplets (R²=0.995). 2×10⁸ nanodroplets/mL (25 wt %) and physiological saline (no droplets) produce the same RF-acoustic amplitude which provides an estimation of the detection limit of the saline nanodroplets in tissues. To benchmark the performance of the nanodroplets, their RF-acoustic signals were compared with two available solid-state nanoparticles that have been used as contrast agents in RF-acoustic imaging studies.

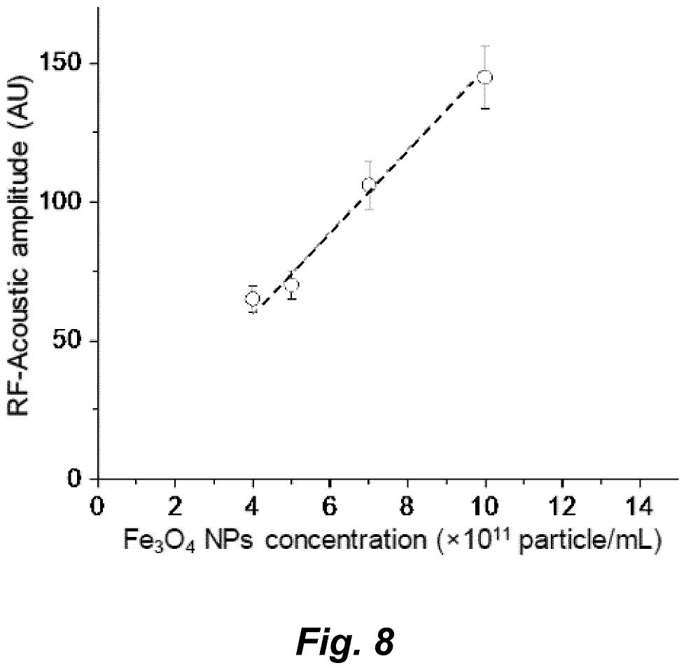

The same tube phantom as in FIG. 2A was used but now filled four of the six tubes with nanodroplets (25 wt % saline, 1×10⁹ nanodroplets/mL), one with gold nanoparticles (20 nm in diameter, 5×10¹¹ nanoparticles/mL), and one with Fe₃O₄ nanoparticles (100 nm in diameter, 5×10¹¹ nanoparticles/mL). FIG. 3D shows the RF-acoustic imaging of the tube phantom and the statistical comparison of their signals. Although gold and Fe₃O₄ nanoparticles are 500 times more concentrated than the nanodroplet solution, the RF-acoustic signals from the nanodroplets were 5.1±0.6 and 3.2±0.7 times stronger than gold nanoparticles and Fe₃O₄ nanoparticles (p=0.00004 and p=0.0002, respectively); since the RF-acoustic signals of Fe₃O₄ nanoparticles are linearly proportional (R²=0.997) to its concentration (FIG. 8), the data suggests that the nanodroplets can potentially produce at least 1500-times and 2500-times stronger RF-acoustic signals than Fe₃O₄ and gold nanoparticles, respectively, when their concentrations are matched with the nanodroplets.

One of the most important motivations for developing UHF-RF-acoustic molecular imaging arises from its potential in deep tissue imaging. To demonstrate this capability, a bovine tissue phantom with six 3-mm-diameter inclusions at different depths beneath the surface was built (FIG. 3E). Nanodroplets (3×10⁹ nanoparticles/mL with 25 wt % saline) mixed with 12% gelatin (1:1 in volume) were filled in the inclusion. The RF waves penetrated up to 5 cm in tissue to reach these inclusions and back to the transducer to mimic deep tissue imaging. Due to the physical size limit of the prototype setup (FIGS. 1A-1D), it was unable to rotate the whole bovine tissue larger than 6 cm in radius. To overcome this constraint and explore the possibility of imaging the nanodroplets at a greater depth (greater than 5 cm), a hole (2.2 cm in diameter) was drilled on a frozen bovine tissue (approximately 9 cm by 9 cm) and developed an agar gel phantom with a Stanford logo inclusion that fits tightly to the drilled hole (FIG. 3F). The smallest feature of this inclusion is around 1-2 mm (the branches of the redwood tree on the logo). The phantom was embedded inside the imaging system. During the tomography, only the agar part of the phantom was rotated. With this setup, deep-tissue UHF-RF-acoustic imaging of saline nanodroplets at 7 cm beneath the surface was demonstrated (FIG. 3F).

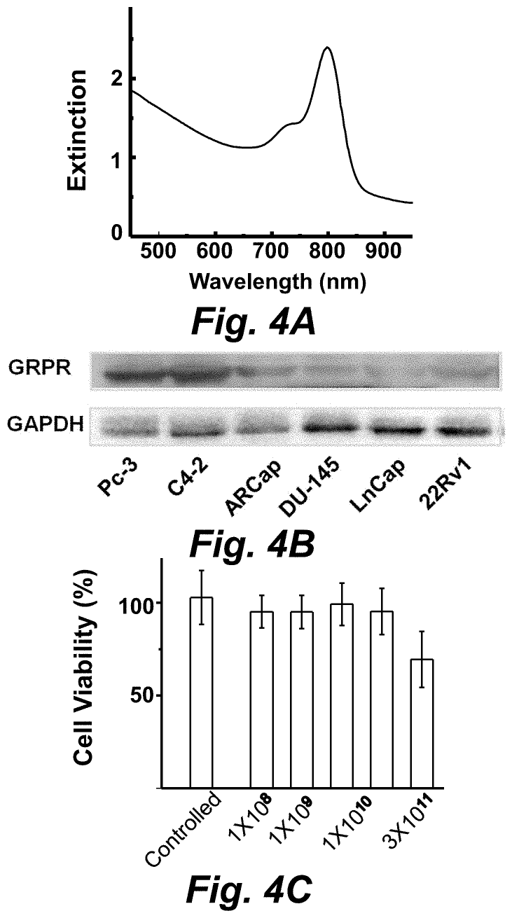

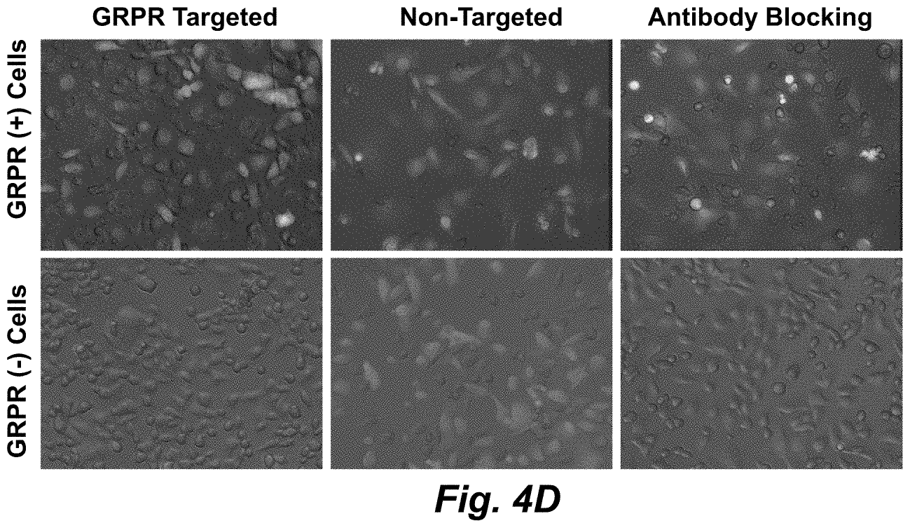

Next, the saline nanodroplets was functionalized to target cancer-associated gastrin-releasing peptide receptor (GRPR) as it is a biomarker overexpressed in different cancers, including prostate, breast, colon, and lung cancers. To couple GRPR-targeting ligands on the surface of nanodroplets, sulfosuccinimidyl 4-(N-maleimidomethyl) cyclohexane-1-carboxylate (Sulfo-SMCC) was used as a linker to conjugate the thiol groups on the nanodroplets with the amine groups on the GRPR antibodies. Additional ICG dyes were also co-conjugated on the surface of nanodroplets using the same chemistry. The ICG dyes enable tracking the nanodroplet distribution within cancer cells in vitro and in mice bearing prostate cancer xenografts by using fluorescent imaging (FIG. 4A).

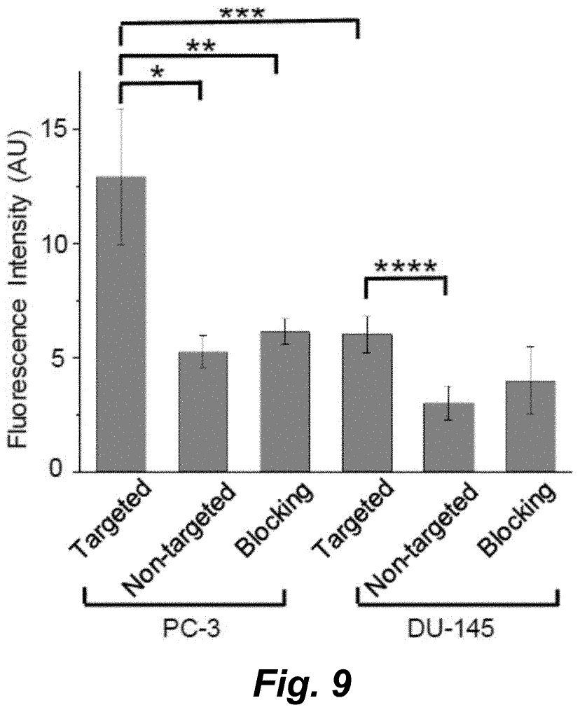

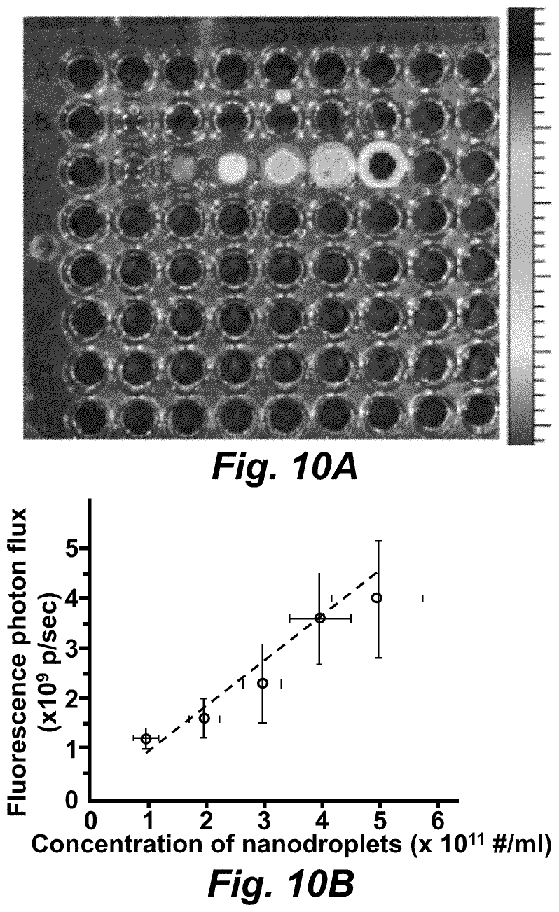

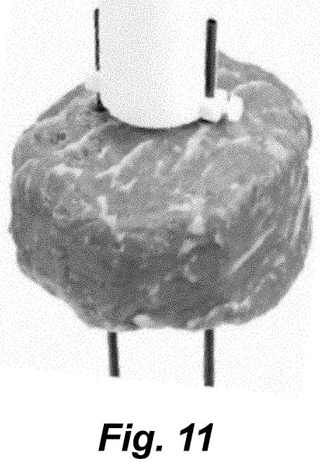

To test targeting specificity of the nanodroplets, the GRPR expression of the common prostate cancer cell lines was first confirmed by western blot analysis (FIG. 4B). PC3 cells that overexpress GRPR (GRPR+) and DU145 cells that do not express significant levels of GRPR (GRPR−) were used as positive and negative controls for non-specific binding, respectively. Conjugation of GRPR antibodies to the nanodroplets does not quench the fluorescent signals of ICG using the epi-fluorescence imaging. To ensure the concentration of nanodroplets used for targeting test does not lead to cytotoxicity in cancer cells, the cell viability was tested with Presto Blue to determine the inhibitory concentration of GRPR targeted nanodroplets. The results show no obvious reduction in cell viability compared to cells without nanodroplets or incubated with the solvent up to 3×10¹¹ nanodroplets/mL. At 3×10¹¹ nanodroplets/mL, the cell viability reduces dramatically to 70% (p=0.0091 relative to cells incubated in the absence of nanodroplets (FIG. 4C). The in vitro GRPR-targeting specificity was tested by fluorescence imaging. Both cell lines were incubated with targeted (GRPR) and non-targeted (PEG) nanodroplets (1×10⁸ nanodroplets for 1×10⁵ cells) for two hours. PC3 cells with the GRPR-targeted nanodroplets show 2.5-fold higher ICG fluorescent signals (p=0.002, n=30 cells analyzed in both cases), compared with the non-targeted (PEG) nanodroplets (FIG. 9). As expected, the negative control cell line DU145 shows much lower fluorescence signals from both targeted and non-targeted nanodroplets due to the relatively low expression of GRPR (p<0.00001, n=30 cells analyzed, FIGS. 4D and 9).

To further confirm molecular specificity, a blocking study on the PC3 cells were performed. GRPRs of PC3 cells were first blocked with GRPR antibodies for 30 minutes prior to incubation with GRPR-targeted nanodroplets. PC3 cells pre-incubated with GRPR antibodies show significantly lower ICG intensities, compared to that without blocking (FIG. 4D) (p=0.006, n=30 cells analyzed). The results validated that the increased uptake of GRPR-targeted nanodroplets in PC3 cells is due to binding of GRPR and confirmed the molecular specificity of the GRPR-targeted nanodroplets.

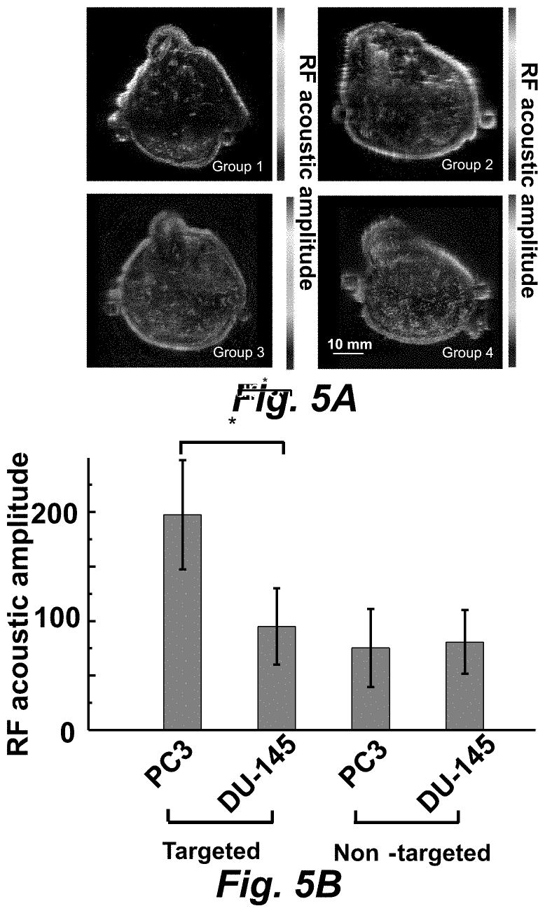

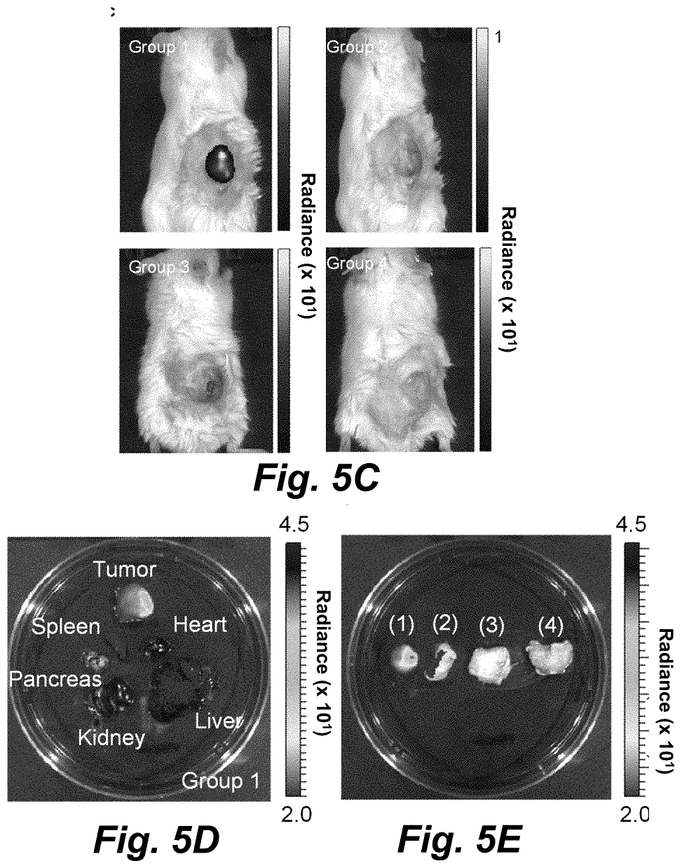

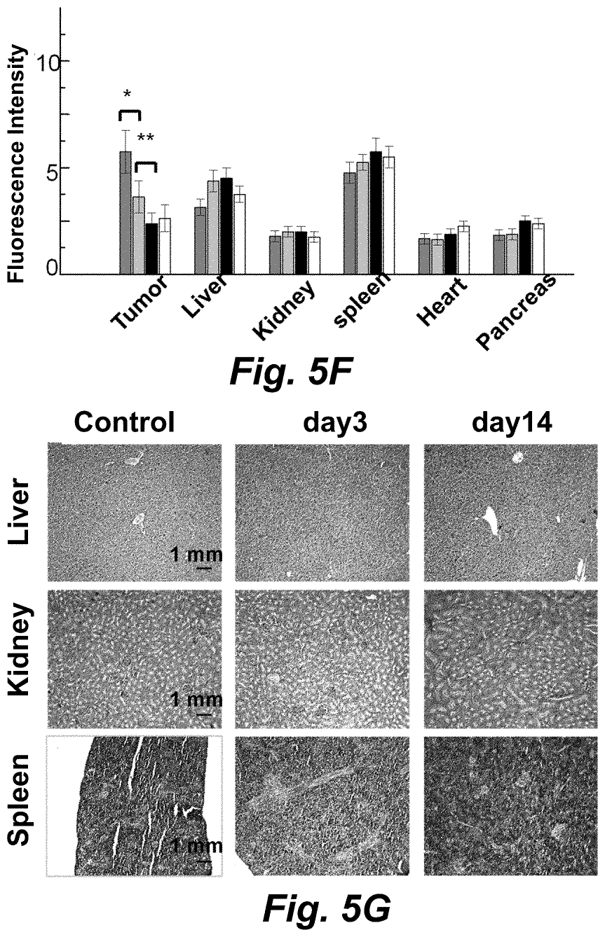

To validate the feasibility of the nanodroplets for UHF-RF-acoustic molecular imaging, in vivo imaging of prostate cancer in a subcutaneous murine tumor model was used. Six-weeks-old male NSG mice (N=20) were randomly divided into four groups: two groups of mice were subcutaneously implanted with GRPR+PC3 cancer cells (groups 1, 3) and two groups were implanted with GRPR-DU145 cells (groups 2, 4) at the mid-dorsal region of each mouse. The subcutaneous tumors were allowed to grow for 2 weeks until they reach approximately 1 cm in diameter before imaging. To track the nanodroplets biodistribution in vivo, the nanodroplets were conjugated with ICG dyes for epi-fluorescent imaging. Before imaging, each tumor-bearing mouse was tail-vein injected with 100 μL of nanodroplets solution (1×10¹¹ nanodroplets/mL). Group 1 (PC3) and group 2 (DU145) mice were injected with GRPR-targeted nanodroplets (GRPR); group 3 (PC3) and group 4 (DU145) mice were injected with non-targeted nanodroplets (PEG). Forty-eight hours after injection, all mice from groups 1 to 4 were imaged using UHF-RF-acoustics. One representative mouse from each group was shown in FIG. 5A, using the same imaging setup as shown in FIGS. 1B-1D.

After the UHF-RF-acoustic molecular imaging, ultrasound imaging of the same mouse in the same field of view used a separated ultrasound system (VisualSonics Vevo 2100). The RF-acoustic images, displayed as color maps, were then overlaid with the ultrasound images for anatomical information. FIG. 5A shows visibly higher RF-acoustic signals from the PC3 tumor with GRPR-targeted nanodroplets (group 1) than tumors in groups 2-4, which indicates the GRPR-specific targeting of the nanodroplets in vivo. By quantitatively comparing among groups 1 to 4, the RF-acoustic signal from group 1 (GRPR-targeted nanodroplets in the PC3 tumors) was statistically higher (2.4±0.3-fold) than those from groups 2-4 (p=0.007, n=5). Right after the RF-acoustic imaging, in vivo epi-fluorescence imaging confirmed the successful delivery and targeting of the nanodroplets in the tumor region. The higher fluorescence signals of group 1 (ICG from GRPR-targeted nanodroplets in the GRPR+PC3 tumors) compared to groups 2-4 corroborated the RF-acoustic imaging results.

Right after the in vivo imaging, key organs (heart, liver, kidney, spleen, pancreas) and tumors of the mice (groups 1-4) were excised for more quantitative analyses of the bio-distribution of nanodroplets (FIGS. 5D and 5E). Among the ICG signals of all the tumors, the ICG signals of group 1 with GRPR-targeted nanodroplets in the PC3 tumors was significantly higher than the rest of the groups (p<0.006 in all cases), and signals in the GRPR-groups 3 and 4 were similar (FIG. 5F).

The signals from groups 3 and 4 may be due to non-specific uptake or enhanced permeability and retention (EPR) effects. The slightly higher ICG signals from group 2 (1.5±0.1-fold, p=0.278) than the non-targeted groups 3 and 4 could result from minimal GRPR expression of the DU145 cells, as observed in the cell culture specificity study (FIG. 4). Quantitative comparison of the ICG signals in tumors and major organs showed that in addition to the tumors, GRPR-targeted and non-targeted nanodroplets mainly accumulated in the spleen and liver, and signals from the rest of organs are close to background noise level (FIG. 5F). The pilot animal toxicity results showed that the nanodroplets did not affect the total blood count, relative to clinical pathology reference ranges (Table 1) and the histology results showed no visible damage to any tissue of the key organs with the utilized dosage at Day 14 (FIG. 5G).

In conclusion, UHF-RF-acoustic imaging is a non-ionizing deep-tissue imaging technique, in particular beneficial to patients who are not suitable to be imaged by traditional imaging techniques using ionizing sources. Thus, an approach to encapsulate high concentration saline into nanodroplet form and used for molecular UHF-RF-acoustic imaging of cancer in living mice using targeted saline nanodroplets was achieved. By controlling the sizes of these nanodroplets, it is possible to stably encapsulated hypertonic saline with a shelf-life for at least 14 days. These nanodroplets have shown to provide about 1500-2500 times stronger RF-acoustic signals than that of concentration-matched iron oxide nanoparticles commonly used in medical imaging, including RF-acoustic imaging. The major core components of these nanodroplets include clinical translatable materials such as saline, perfluorodecalin, and Pluronic F-68, all of which have been approved by the Food and Drug Administration (FDA) for medical use. The finding that perfluorocarbon liquid can stabilize high ionic liquid into a stable nanodroplet can be useful to the fields that are interested in developing nanocarriers to deliver high ionic prodrug.

One aspect of the disclosure encompasses embodiments of a composition comprising a double emulsion nanodroplet that generates a detectable acoustic signal when irradiated by a radio frequency, wherein the nanodroplet can comprise (i) a high ionic salt solution liquid core and (ii) a fluorinated shell encapsulating the high ionic salt solution liquid core and having an outer surface, wherein the fluorinated shell can be both hydrophobic and oleophobic.

In some embodiments of this aspect of the disclosure, the composition can further comprise a non-ionic surfactant.

In some embodiments of this aspect of the disclosure, the salt solution can be an aqueous solution of a salt selected from the group consisting of sodium chloride (NaCl), sodium hydroxide (NaOH), potassium iodide (KI), potassium chloride (KCl), magnesium chloride (MgCl₂), and calcium chloride (CaCl₂).

In some embodiments of this aspect of the disclosure, the salt solution can be a solution of sodium chloride.

In some embodiments of this aspect of the disclosure, the fluorinated shell can comprise a perfluoroalkane.

In some embodiments of this aspect of the disclosure, the perfluoroalkane is selected from the group consisting of: perfluoropentane, perfluorohexane, perfluoro-15-crown-5-ether, and perfluorodecalin.

In some embodiments of this aspect of the disclosure, the composition can further comprise a reactive group disposed at the outer surface of the fluorinated shell.

In some embodiments of this aspect of the disclosure, reactive group can be a reactive thiol group.

In some embodiments of this aspect of the disclosure, the reactive thiol group is 1,2-distearoyl-sn-glycero-3-phosphoethanolamine-N-(polyethylene glycol)-thiol (DSPE-PEG₂₀₀₀-SH).

In some embodiments of this aspect of the disclosure, the composition can further comprise at least one functional moiety conjugated to the reactive thiol group, wherein the functional moiety can be a detectable label, a ligand having selective affinity for a cell or a biomarker of a cell, an immunoglobulin or fragment thereof having selective affinity for a cell or a biomarker of a cell, a protecting molecule that reduces immunogenicity of the nanodroplet, or a pharmaceutically active agent.

In some embodiments of this aspect of the disclosure, the detectable label can be a fluorescent dye.

In some embodiments of this aspect of the disclosure, the at least one functional moiety can be conjugated to the reactive thiol group by a linker.

In some embodiments of this aspect of the disclosure, the linker can be sulfosuccinimidyl 4-(N-maleimidomethyl) cyclohexane-1-carboxylate (Sulfo-SMCC).

In some embodiments of this aspect of the disclosure, the fluorescent dye can be indocyanine green (ICG).

In some embodiments of this aspect of the disclosure, the ligand having selective affinity for a cell or a biomarker of a cell can selectively bind to a cell receptor.

Another aspect of the disclosure encompasses embodiments of a method of generating an acoustic image comprising administering to an animal or human subject a pharmaceutically acceptable composition comprising a nanodroplet according to any one of claims of the disclosure; irradiating the animal or human subject with a radio frequency that generates an acoustic signal from the nanoparticle, detecting the acoustic signal; and generating an image of the animal or human subject showing the location of the nanoparticle in the animal or human subject.

Yet another aspect of the disclosure encompasses embodiments of a method of synthesizing an encapsulated high ionic concentration saline nanodroplet, the method comprising the steps: (a) mixing an activated perfluoroether with poly(propylene glycol)-block-poly(ethylene glycol)-block-poly(propylene glycol)bis(2-aminopropyl ether; (b) synthesizing a first emulsion by sonicating a mixture of the product of step (a), a perfluoroalkane, and an aqueous salt solution to generate a first emulsion; and (c) generating a double emulsion by sonicating a mixture of the product of step (c) and a non-ionic surfactant, thereby forming a population of encapsulated saline nanodrops.

In some embodiments of this aspect of the disclosure, the non-ionic surfactant can be Pluronic F-68.

In some embodiments of this aspect of the disclosure, the method can further comprise the step of functionalizing the encapsulated saline nanodroplets with a reactive group.

In some embodiments of this aspect of the disclosure, the reactive group can be a reactive thiol group.

In some embodiments of this aspect of the disclosure, the reactive thiol group can be 1,2-distearoyl-sn-glycero-3-phosphoethanolamine-N-(polyethylene glycol)-thiol (DSPE-PEG₂₀₀₀-SH).

In some embodiments of this aspect of the disclosure, the method can further comprise at least one functional moiety conjugated to the reactive thiol group, wherein the functional moiety can be a detectable label, a ligand having selective affinity for a cell or a biomarker of a cell, an immunoglobulin or fragment thereof having selective affinity for a cell or a biomarker of a cell, a protecting molecule that reduces immunogenicity of the nanodroplet, or a pharmaceutically active agent.

In some embodiments of this aspect of the disclosure, the detectable label is a fluorescent dye.

In some embodiments of this aspect of the disclosure, the at least one functional moiety is conjugated to the reactive thiol group by a linker.

In some embodiments of this aspect of the disclosure, the linker can be sulfosuccinimidyl 4-(N-maleimidomethyl) cyclohexane-1-carboxylate (Sulfo-SMCC).

In some embodiments of this aspect of the disclosure, the fluorescent dye can be indocyanine green (ICG).

In some embodiments of this aspect of the disclosure, the ligand having selective affinity for a cell or a biomarker of a cell specifically binds to a cell receptor.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

### Example 1

### Materials:

All chemicals in this study were used as received: Krytox 157 FSL (DuPont), polyethylene glycol-amine (Sigma-Aldrich), methoxyperfluorobutane (Sigma-Aldrich), oxalyl chloride (Sigma-Aldrich), poly(propylene glycol)-block-poly(ethylene glycol)-block-poly(propylene glycol)bis(2-aminopropyl ether) (Mn: 900, Sigma-Aldrich), anhydrous dichloromethane (Sigma-Aldrich), sodium chloride (Sigma-Aldrich), sodium hydroxide (Sigma-Aldrich), potassium iodide (Sigma-Aldrich), potassium chloride (Sigma-Aldrich), magnesium chloride (Sigma-Aldrich), and calcium chloride (Sigma9-Aldrich), perfluoro-n-hexane (Fluoromed), perfluoropentane (Synquest), perfluoro-15-crown-5-ether (Synquest), and perfluorodecalin (Synquest), soybean oil (Sigma-Aldrich), Pluronic F-68 (Poloxamer 188, Fisher), soybean oil (Sigma-Aldrich), Brij 93 (Sigma-Aldrich), 1,2-Dipalmitoyl-sn-glycero-3-phosphocholine (DPPC, Avanti), 1,2-Dipalmitoyl-sn-glycero-3-phosphate (DPPA, Avanti), 1,2-distearoyl-sn-glycero-3-phospho-ethanolamine-N-[thiol(poly-ethylene glycol)-2000] (DSPE-PEG2K—SH, NanoCS), anti-gastrin-releasing peptide receptor antibody (AbCam), Glyceraldehyde 3-phosphate dehydrogenase (GAPDH) loading control monoclonal antibody (Thermo Fisher Scientific), Thiol quantification assay kit (AbCam), sulfosuccinimidyl 4-(N-maleimidomethyl)cyclohexane-1-carboxylate (Sulfo-SMCC, Thermo Scientific), Indocyanine green (ICG) maleimide (AAT Bioquest), growth factor reduced Matrigel (Corning), Phosphate-buffered saline (PBS, Thermo Fisher Scientific), Presto Blue (Invitrogen), anti-GRPR antibody (AbCam).

### Example 2

### Synthesis of Perfluoro-Surfactant:

The perfluoro-surfactant for stabilizing the primary emulsion (NaCl_{(aq)}/perfluorocarbon liquid) was synthesized using a two-step reaction to PEGylate Krytox 157 FSL, a carboxylic acid functionalized purfluoroether (Holtze et al. (2008) Lab on a chip 8: 1632-1639). The carboxylic acid of the Krytox 157 FSL was first converted to acid chloride and then linked with polyoxyethylene (PEG) diamine via nucleophilic addition/elimination reaction. Briefly, under a nitrogen purge, Krytox 157 FSL was added to a flask containing the fluorinated solvent methoxyperfluorobutane. Oxalyl chloride was then added (in 10:1 molar excess to the Krytox 157 FSL) to activate its terminal carboxylic groups. The flask was refluxed and stirred for 24 hr at 50° C. under a nitrogen purge. The residue oxalyl chloride in the resulting mixture was removed using a rotary evaporator. Poly(propylene glycol)-block-poly(ethylene glycol)-block-poly(propylene glycol)bis(2-aminopropyl ether) was then dissolved in a mixture of anhydrous dichloromethane and methoxyperfluorobutane (2.3:1 volumetric ratio) and added into the activated Krytox 157 FSL. Similar experimental conditions of the first step were used for the reaction. The resulting copolymer, called Krytox-PEG, was concentrated using a rotary evaporator to remove all the solvent.

### Example 3

### Synthesis of Nanodroplets:

Nanodroplets containing a high concentration NaCl(aq) were synthesized with a double emulsion method via ultrasonic agitation. Krytox-PEG (0.6 mg/uL PFC) was first dissolved in 500 μL of perfluoro-n-hexane in a 5 mL microcentrifuge tube. Next, 400 μL of a NaCl(aq) (25%) solution was added drop-wise to the PFC phase under sonication using a microtip (Branson 450, 20 kHz, 3.2 mm diameter, 250 W/cm2, continuous mode) and continued for another 30 seconds after the addition. The mixture was emulsified in an ice bath. In a 50 mL Falcon conical centrifuge tube, the primary emulsion was then added drop-wise to 4 ml of Pluronic F-68 (5 mg/mL) under sonication using the same microtip (250 W/cm2, continuous mode) and continued for another 30 seconds after the addition. The resulting double emulsion was centrifuged at 50 g for 10 minutes at 4° C. The supernatant was collected and dialyzed (Slide-A-Lyzer, ThermoScientific, 10K MWCO) against deionized water for two days to remove free NaCl. During the dialysis, water was replaced every 8 hours. After the dialysis, the sample was collected and stored at 4° C. The same protocol was applied for all the rest perfluorocarbon liquid/saline nanodroplets mentioned in the main text (perfluoropentane, perfluorohexane, perfluoro-15-crown-5-ether, and perfluorodecalin shell saline nanodroplets). The nanodroplets with diameters 450 nm and 800 nm were produced with the same above-mentioned conditions except for the concentrations of Krytox-PEG (4 mg/mL PFC for both sizes), Pluronic F-68 (2 mg/mL for 450 nm and 1 mg/mL for 800 nm), and the power of sonication (200 W/cm2 for 450 nm and 150 W/cm2 for 800 nm). The referenced saline-soybean oil nanodroplets in aqueous solution were prepared by the same method except that we used soybean oil and Brij 93 as the oil phase and surfactants respectively for the first inversed emulsion.

### Example 4

### Thiol Functionalization of Nanodrops:

Thiol functional groups were added to the surface of preformed saline nanodroplets utilizing the post-insertion method as described in Messerschmidt et al., (2008) Bioconj. Chem. 19: 362-369, incorporated herein by reference in its entirety. Briefly, 10 mg of DPPC:DSPE-PEG2K—SH:DPPA (with a molar ratio of 0.8:0.15:0.05) was dissolved in 2 mL of mixture of chloroform-methanol solution (with a volume ratio of 2:1). The solvent of the mixture was removed by evaporation using a rotary evaporator. The phospholipid film was hydrated using 2 mL of Pluronic F-68 solution (2 mg/mL). The phospholipid suspension was then slowly added into a beaker containing the nanodroplet solution while the solution was gently rotated by a digital shaker (10 rpm, room temperature) and continued to rotate at the same speed for 4 hrs. The nanodroplets were then purified and solvent exchanged twice by desalting columns (PD-10, Sephadex G-25, GE).

### Example 5

### GRPR Antibody and ICG Dye Conjugation:

An anti-GRPR antibody (AbCam) was chosen as a binding moiety for preparing GRPR-targeting nanodrops. In addition, ICG dyes were also conjugated to the nanodroplets for both cell fluorescent imaging and in vivo epi-fluorescent imaging.

Antibodies were conjugated to partial thiol groups of PEGs on the nanodroplet surface via a molecular linker, Sulfo-SMCC; at the same time, ICG dyes were conjugated on other partial thiol groups of PEGs on the nanodroplet surface. First, antibody (1 mg/mL) was added to Sulfo-SMCC solution (20-fold molar excess, in PBS, 4.8 mg/mL, pH 7.2). The mixture was incubated for 2 hr at 4° C. The mixture was purified twice to remove free Sulfo-SMCC molecules using a centrifuge desalting column (Bio-Spin 6 Columns, Bio-Rad). Sulfo-SMCC conjugated antibodies and ICG-maleimide were added to nanodroplet solution (10¹¹ nanodrops/mL in PBS pH7.2; molar ratio of antibodies:ICG:nanodroplets was 1:9:7.5) and incubated for 2 hr at 4° C. After the reaction, the nanodroplets were purified and solvent exchanged twice with a desalting column (PD-10, Sephadex G-25, GE).

### Example 6

### Characterization of Nanodroplets:

The optical absorption of the ICG-conjugated nanodroplets was characterized with ultraviolet to visible (UV-Vis) extinction spectroscopy. Extinction spectra were collected from a 0.1 mL nanodroplet suspension in a Microplate Reader (BioTek, Synergy) at room temperature. The fluorescent intensities of the ICG nanodroplet were characterized with epi-fluorescence imaging using IVIS spectrum imaging system (PerkinElmer). The fluorescent images after 60 secs of exposure were recorded with the excitation filter centered at 745 nm and the emission filter centered at 840 nm. The florescent intensities were quantified by Living Image® 4.5 software. The extracted fluorescent intensities were plotted as a function of optical density (OD), and fitted with linear regression. The slopes of the linear regression fitting curves were used as the scaling factors to quantify the concentration of nanodroplets in the bio-distribution test (FIG. 5).

The average size of the nanodroplets was measured with a dynamic light scattering tool (DLS, Zetasizer Nano ZS, Malvern) at room temperature and the size distribution of nanodroplets was determined by the polydispersity value (PDI). The concentration of the nanodroplets was measured by nanoparticle tracking analysis (Nanosight NS300, Malvern) at 25° C. The morphology of the nanodroplets was assessed with cryo-transmission electron microscopy (Cryo-TEM) imaging (FEI Titan Krios at 270 kV with a Gatan K2 Summit direct detection camera in counting mode with 8 second exposure per frame). The cryo-TEM sample was prepared by first drop-casting the sample (1×10¹⁰ nanodrops/mL in water) on a lacey carbon coated copper TEM grid, then froze with an automatic plunge freezer (EM GP, Leica, 6 second plunge time, 95% humidity). The sample was saved and transferred to TEM under the cooling of liquid nitrogen.

### Example 6

### Functionalization Confirmation and Quantification:

The concentration of thiol groups on the nanodroplets were confirmed and quantified by a fluorometric thiol quantification assay (FIG. 6).

### Example 7

### RF-Acoustic Signal Characterization:

To characterize RF-acoustic signal of the nanodrops, a 3D-printed a sample holder was designed (FIG. 7) to vertically hold 7 polyethylene tubes (1 mm in diameter). The tubes were placed concentrically to the center of rotation with a radius of 1.5 cm to ensure that each sample would be exposed to the same power of RF wave during the tomography. Nanodroplet solutions and a PBS control solution (physiological saline, 0.9% NaCl) were filled in the tubes, sealed, and placed on the holder for imaging.

To prepare the bovine tissue phantom with tube inclusions, the bovine tissue was first froze at −20° C. for 4 hrs. The frozen tissue was drilled with an M3 drill bit to create a vertical tube inclusion (with a diameter of about 3 mm). The nanodroplet solutions were mixed with 12% gelatin (1:1 volume ratio) and filled to the holes in the tissue. A 3D-printed holder was built to hold the tissue vertically. During imaging, the whole tissue was rotated within the imaging plane.

To prepare a logo phantom, a mode of a logo was 3D-printed. The mold was used to prepare an agar (5%) mode with a logo inclusion (with a diameter of about 3 cm). The nanodroplet solutions were mixed with 12% gelatin (1:1 volume ratio) and filled in the space of the inclusion. A 3D-printed holder was built to hold the agar phantom vertically.

To prepare the bovine tissue enclosure, the same freezing protocol was used except that a hole saw (diameter: 3 cm) was used to drill a hole (with a diameter of about 3.1 cm) to fit the agar gel phantom from the previous step. The tissue was placed in the imaging plane but only the agar gel phantom is rotated during the tomography.

### Example 8

### Cell Culture:

Human prostate cancer cell lines, PC3-GFP and DU145-GFP cells were purchased from American Type Tissue Collection (ATCC) and cultured on collagen-coated flasks (BD Biosciences) in RPMI 1640 supplemented with 10% heat-inactivated fetal bovine serum. The cultures were maintained in a humidified incubator with 5% CO₂/95% air at 37° C. The cell lines were authenticated at Stanford functional genomics facility using Short Tandem Repeat (STR) profiling. The cell lines were tested for mycoplasma contamination upon received, after thawing, and monthly during culture using MycoAlert Mycoplasma Detection Kit (Lonza).

### Example 9

### Cell Culture Uptake of GRPR-Targeted Nanodroplets:

1×10⁴ cells PC3 and DU145 cells labeled with green fluorescence protein (PC3-GFP and DU145-GFP cells) were plated in a 96-well plate. Twenty-four hours after seeding, the cell media were removed, and the cells were washed twice with PBS. Then, the cells were incubated with 1×10⁹ nanodroplets in the cell culture medium for 12 hours. After that, cells were imaged by a fluorescent microscope (EVOS FL cell imaging system, Thermo Fisher Scientific) with 20× objective lens, and its GFP filter set and ICG filter set.

### Example 10

### Western Blot:

The expression of GRPR on 6 prostate cancer cell lines (PC3, C4-2, ARCap, DU145, LnCap, 22Rv1) were evaluated with western blotting with a rabbit polyclonal anti-GRPR antibody using cell lysates prepared from prostate cancer cells. The protein concentration was monitored with a Glyceraldehyde 3-phosphate dehydrogenase (GAPDH) loading control monoclonal antibody (37 KDa).

### Example 11

### Cell Viability Test:

PC3 cells were plated in triplicate for each nanodroplet concentrations (1×10⁸, 1×10⁹, 1×10¹⁰, 3×10¹¹ and a control 0.9% saline) at a density of 1×10⁴ cells per well in a 96-well plate. Cells were allowed to grow in RPMI 1640 media supplemented with 10% heat-inactivated fetal bovine serum for 24 hr, then the wells were washed with PBS. The 100 μL of mixture of nanodroplets in the medium was added to the wells and incubate at cell incubator for 24 hrs. Nanodroplets and medium was then removed from the wells and replaced with 100 μL of medium and 10 μL of Presto Blue. The plate was incubated at 37° C. for 30 min before fluorimeter reading at 590 nm (excitation 560 nm, Synergy 4, BioTek).

### Example 12

### RF-Acoustic Imaging System:

A prototype RF-acoustic tomography imaging system (with main parts from Endra) was used to measure the RF-acoustic signal from the phantom and the mice. The samples were mounted on a rotational stage that can rotate 360 degrees with 10 degrees of intervals. During the imaging, the samples were rotated at 10-degree intervals. At each angle, 4095 pulses were recorded and averaged to improve the signal-to-noise ratio. A nanosecond 433 MHz RF pulse (pulse duration 160 ns, repetition rate of 1500 Hz) was broadcasted through a pair of customized horn antennas at the near field to the phantom. The average energy density of a pulse at the target site was measured to be 1 mJ/cm², which is within the safety limit of tissue exposure. A 3.5 MHz linear array ultrasound transducer (Acuson L382, bandwidth approximately 75%, depth of focus 82 mm) measures the generated acoustic waves. Acoustic signals were recorded by an ultrasound imaging system. A filtered back projection algorithm was used to reconstruct each image. The reconstruction software was provided by Endra Inc.

### Example 13

### Animal Studies:

Healthy male NSG mice (Jackson laboratory) at age 6 weeks were used. A prostate cancer in mouse model was developed by subcutaneously injecting 100 μL of 5×10⁶ of prostate cancer cells mixed with 1:1 volume ratio of growth factor reduced Matrigel (Corning) into the right flank of each mouse. The tumor was allowed to grow to about 1 cm³ before imaging. Before imaging, mice were anesthetized with 2% isoflurane at 2 liters min⁻¹ of oxygen flow and confirmed with tail pinch. On hundred microliters of nanoparticles/phosphate-buffered saline solution (nanodroplets in PBS, 1×10¹¹ nanodrops/mL) were injected to the mice through tail veins.

### Example 14

### In Vivo RF-Acoustic/Ultrasound Imaging:

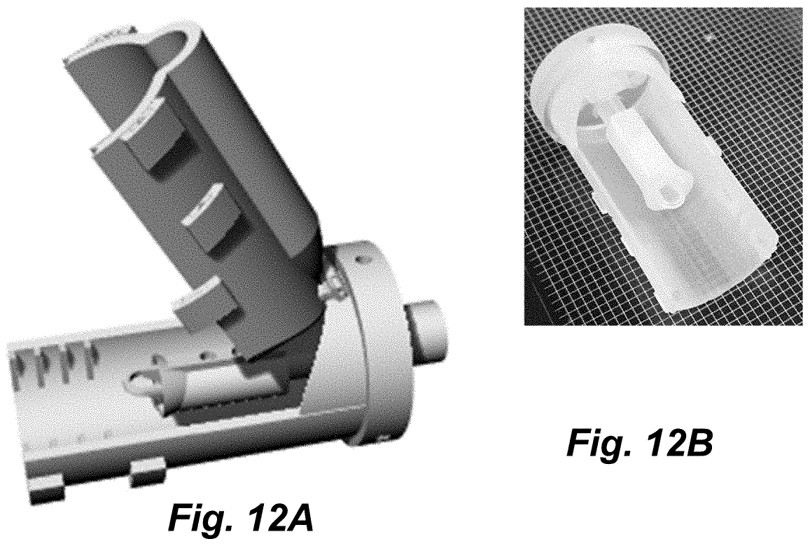

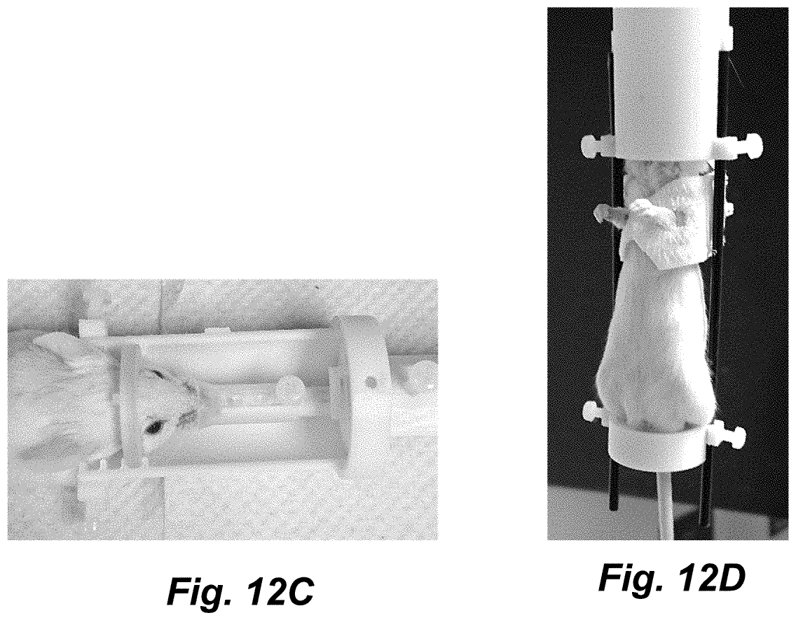

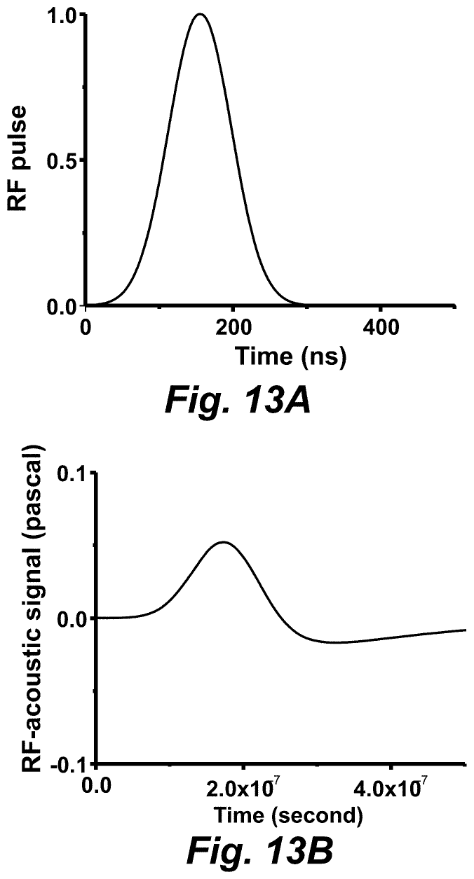

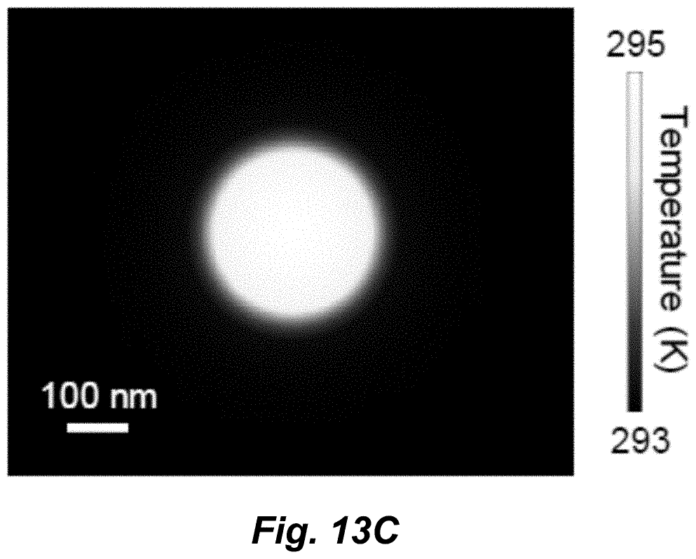

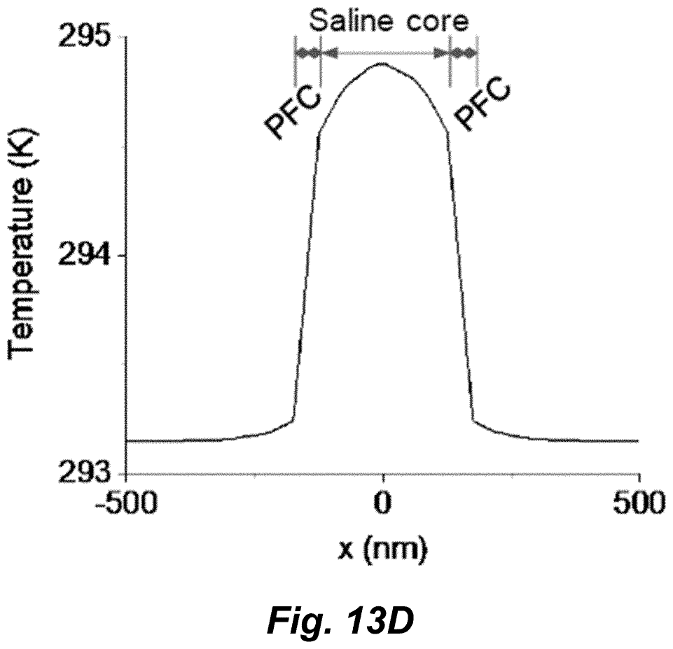

For in vivo imaging, RF-acoustic imaging and ultrasound imaging were recorded separately. The same RF-acoustic prototype system was used with a home-made mouse holder (FIGS. 12A-12D). The mouse was held vertically (perpendicular to the imaging plane of the ultrasound transducer) while anesthetized with 2% isofluorane. The mouse body below the neck was immersed in the water at a constant temperature (37° C.). The same imaging condition was used for recording the ultrasound imaging, except that an additional ultrasound imaging system (VisualSonic 2100) with an imaging transducer (VisualSonic LZ250, central frequency: 21 MHz) was used to collect ultrasound imaging of the mouse on the same animal holder. The recorded images are then registered with RF-acoustic images by the alignment makers on the images.

### Example 15

### Nanodroplet Toxicity Tests:

Histology studies were conducted of tissue, complete blood count, and chemistry panel in three additional three groups of healthy NSG mice (each group with 5 mice). The same amount of the nanodroplets used in imaging were injected through the tail vein to the first and second groups of mice, and the same volume of PBS to the third group as a control. For the first and second groups, their vitals and behavior immediately were recorded after particle administration. The first group of the mice were sacrificed at 3 days post-injection to collect tissues (liver, kidney, spleen, pancreas, heart) and blood. The second and third groups were sacrificed at 14 days post-injection. The toxicity results show that the nanodroplets neither affect the blood count (blood test assay) nor damage any tissue of the main organs at the dosage used (Table 1).

### Example 16

### Quantification of Bio-Distribution of Nanodroplets:

The nanodroplet distribution in tissue of the main organs was quantified with epi-fluorescence imaging. For the bio-distribution, mice were sacrificed 48 hours post-injection of the nanodroplets. Epi-fluorescence imaging of the excised organs was carried out using an IVIS spectrum imaging system (PerkinElmer) with an excitation filter centered at 745 nm and an emission filter centered at 840 nm with 60 seconds of exposure time. The images were quantitatively analyzed using Living Image 4.5 software using the radiant efficiency as the read-out.

### Example 17

### Numerical Simulations:

Numerical simulations used finite-element-method (FEM, COMSOL) solvers. To obtain the RF absorption of the nanodroplets, Matlab was used to calculate the ionic absorption of the nanodroplets. The surface temperature of the nanodroplets and the generated acoustic signals are predicted using COMSOL.

In the COMSOL simulation, it was assumed that a single nanodroplet immersed in saline with 0.9 wt % concentration was excited by a RF pulse. The nanodroplet has a saline core 25 wt % with a diameter of 250 nm and a perfluorocarbon shell with a thickness of 50 nm. These dimensions are extracted from the experimental results, confirmed with cryo-transmission electron microscopy imaging. A heat transfer module was used to estimate the temperature profile of the nanodroplet. An incoming pulse with 100 ns (FWHM) was absorbed by the nanodroplet; the temporal profile of this incoming pulse was assumed to be Gaussian. Due to the RF absorption of nanodroplets compared to that of physiological saline at 433 MHz, during the RF pulse irradiation, the nanodroplet is gradually heated up and its temperature elevates. At the same time, the heat from the nanodroplet diffuses into the surrounding water. Such temperature elevation and heat leaking out of the nanodroplet is a time-dependent and competing process. During this process, the heated nanodroplet acts as a heat source.

A temporal resolution of 2 ns was used during the simulation and within each time step the absorbed heat inside the nanodroplet was assumed to be uniformly distributed across the entire volume of the nanodroplet. A temperature of 293.15 K was used as the initial temperature, and an open boundary is assumed as the outer physiological saline domain. The obtained position- and time-dependent temperature profile was used and coupled to the time-dependent secondary differential equation that calculates the generated acoustic signal.

### Example 18

### Data and Statistical Analysis:

For data analysis, MATLAB was used to process the images acquired with the Vevo imaging system. The ultrasound images are shown in dB scale, and the RF-acoustic images in linear scale. The two-dimensional (2D) tube phantom images shown in FIG. 2 are the maximum intensity projections of the 3D volumetric images to the plane of lateral axis and elevational axis of the transducer. Data plot, average, and standard deviation were computed in Origin pro 2009.

To test the nanodroplet uptake in the in vitro study, the mean and standard deviation of fluorescent intensities was calculated by first summing the fluorescent intensities of ICG from 30 cells (red channel) and then normalized it with the cell footprint. This footprint was defined as the area covered with GFP (green channel), indicating the viability of the cell. Image J was used here for signal analysis. For bio-distribution of the nanodroplets in the in vivo studies, the fluorescent intensities within the region of interest were summed. The region of interest was identified by the footprint of each organ from the photographic images. The summation was then normalized with the footprint to obtain the mean and standard deviation. In both studies, the two-tailed p-value was calculated using an unpaired student t-test to determine the significance. Data was significant with p<0.05.

### Example 19

TABLE 1
Complete blood count.
Result

| | Mouse | Mouse | Mouse | Mouse | Mouse | | Reference |
|---|---|---|---|---|---|---|---|
| Tests | 1 | 2 | 3 | 4 | 5 | Averaged | Range |
| WBC | 3.78 | 5.45 | 6.35 | 8.86 | 11.01 | 7.09 ± 1.43 | 5.5-9.3^{a} |
| (K/μL) | | | | | | | |
| RBC | 9.25 | 9.11 | 9.1 | 8.6 | 8.79 | 8.97 ± 0.133 | 7.0- 8.8 |
| (M/μL) | | | | | | | |
| HGB | 14.1 | 14.3 | 13.9 | 12.9 | 13.6 | 13.76 ± 0.273 | 13.7-16.4 |
| (gm/dL) | | | | | | | |
| HCT(%) | 49.8 | 46.4 | 46.5 | 44.4 | 45.4 | 46.5 ± 1.016 | 39.0-47.0 |
| MCV | 53.8 | 50.9 | 51.1 | 51.6 | 54.6 | 52.4 ± 0.843 | 52.0-68.7 |
| (fL) | | | | | | | |
| MCH | 15.2 | 15.7 | 15.3 | 15 | 15.5 | 15.34 ±0.135 | 18.4-19.6 |
| (pg) | | | | | | | |
| MCHC | 28.3 | 30.8 | 29.9 | 29.1 | 30 | 29.62 ± 0.476 | 34.0-36.0 |
| (g/dl) | | | | | | | |
| Platelet | 1633 | 1261 | 844 | 1595 | 1435 | 1353.6 ± 160.318 | 675-1338 |
| Count | | | | | | | |
| (K/μL) | | | | | | | |
| RDW | 20.2 | 19.5 | 22.2 | 20.9 | 19 | 20.36 ± 0.627 | 16.9-23.5 |
| (%) | | | | | | | |
| MPV | 6.4 | 7 | 7 | 7.1 | 6.5 | 6.8 ± 0.162 | 6.5-8 |
| (fl) | | | | | | | |
| PCT | 1.05 | 0.88 | 0.59 | 1.14 | 0.93 | 0.918 ± 0.105 | 0.3-0.9 |
| (%) | | | | | | | |
| Reticulocyte | 5.11 | 6.07 | 6.62 | 6.41 | 3.66 | 5.574 ± 0.608 | 3.00-5.8 |
| Count | | | | | | | |
| (%) | | | | | | | |
| Neutrophils | 85 | 29 | 32 | 35 | 40 | 44.2 ± 11.583 | 50-75^{b} |
| (%) | | | | | | | |
| Lymphocytes | 10 | 49 | 51 | 50 | 43 | 40.6 ± 8.69 | 10.5-40^{b} |
| (%) | | | | | | | |
| Monocytes | | 22 | 17 | 15 | 17 | 14.4 ± 3.96 | 5-14.5 |
| (%) | | | | | | | |

^{a}Stanford VSC Clinical Lab mouse reference value.
^{b}Reference ranges adapted from Knibbe-Hollinger et al., ((2015) Biol. Open 4: 1243-1252).

NSG mice (n=5 per group) were tail-vein injected with nanodroplets and hematological and biochemical measurements were taken at 2 weeks post injection. Values represent the mean±standard deviation. Clinical pathology reference ranges are from The Jackson Laboratory unless otherwise indicated.

WBC: white blood cells; RBC: red blood cells; HGB: hemoglobin; HCT: hematocrit; MCV: mean corpuscular volume; MCHC: mean corpuscular hemoglobin concentration; RDW: red cell distribution width; MPV: mean platelet volume; PLT: Platelet count; and PCT: plateletcrit ALT: alanine transaminase; Alk Phos: alkaline phosphatase; BUN: blood urea nitrogen; T. Protein: total protein; TIBC: total iron-binding capacity.

TABLE 2
Thermophysical properties of perfluorocarbon liquid-

| | Perfluoro- | Perfluoro- | Perfluoro- | Perfluoro-15- |
|---|---|---|---|---|
| Property | pentane | hexane | decalin | crown-5-ether |
| Molecular | 288.04 | 337.90 | 461.90 | 580.01 |
| weight | | | | |
| (g mol⁻¹) | | | | |
| Density | 1.63 | 1.68 | 1.93 | 1.78 |
| (kg m⁻³) | | | | |
| Boiling point | 28.00-36.00 | 58.00-60.00 | 144.00 | 145.00 |
| (° C.) | | | | |
| Vapor | 83.99 | 29.41 | 1.02 | 0.92 |
| pressure | | | | |
| (kPa) | | | | |
| Water | 4.00 × 10⁻⁶ | 2.70 × 10⁻⁷ | 9.90 × 10⁻⁹ | No data |
| solubility | | | | |

What is claimed:

1. A composition comprising a double emulsion nanodroplet that generates a detectable acoustic signal when irradiated by a radio frequency, wherein the nanodroplet comprises (i) a high ionic salt solution liquid core and (ii) a fluorinated shell encapsulating the high ionic salt solution liquid core and having an outer surface, wherein the fluorinated shell is both hydrophobic and oleophobic.

2. The composition of claim 1, further comprising a non-ionic surfactant.

3. The composition of claim 1, wherein the salt solution is an aqueous solution of a salt selected from the group consisting of sodium chloride (NaCl), sodium hydroxide (NaOH), potassium iodide (KI), potassium chloride (KCl), magnesium chloride (MgCl₂), and calcium chloride (CaCl₂).

4. The composition of claim 3, wherein the salt solution is a solution of sodium chloride.

5. The composition of claim 1, wherein the fluorinated shell comprises a perfluoroalkane.

6. The composition of claim 5, wherein the perfluoroalkane is selected from the group consisting of: perfluoropentane, perfluorohexane, perfluoro-15-crown-5-ether, and perfluorodecalin.

7. The composition of claim 1, further comprising a reactive group disposed at the outer surface of the fluorinated shell.

8. The composition of claim 1, wherein the reactive group is a reactive thiol group.

9. The composition of claim 8, wherein the reactive thiol group is 1,2-distearoyl-sn-glycero-3-phosphoethanolamine-N-(polyethylene glycol)-thiol (DSPE-PEG₂₀₀₀-SH).

10. The composition of claim 8, further comprising at least one functional moiety conjugated to the reactive thiol group, wherein the functional moiety is a detectable label, a ligand having selective affinity for a cell or a biomarker of a cell, an immunoglobulin or fragment thereof having selective affinity for a cell or a biomarker of a cell, a protecting molecule that reduces immunogenicity of the nanodroplet, or a pharmaceutically active agent.

11. The composition of claim 10, wherein the detectable label is a fluorescent dye.

12. The composition of claim 10, wherein the at least one functional moiety is conjugated to the reactive thiol group by a linker.

13. The composition of claim 12, wherein the linker is sulfosuccinimidyl 4-(N-maleimidomethyl) cyclohexane-1-carboxylate (Sulfo-SMCC).

14. The composition of claim 11, wherein the fluorescent dye is indocyanine green (ICG).

15. The composition of claim 10, wherein the ligand having selective affinity for a cell or a biomarker of a cell specifically binds to a cell receptor.

16. A method of generating an acoustic image comprising administering to an animal or human subject a pharmaceutically acceptable composition comprising a nanodroplet according to any one of claims 1-15, irradiating the animal or human subject with a radio frequency that generates an acoustic signal from the nanoparticle, detecting the acoustic signal; and generating an image of the animal or human subject showing the location of the nanoparticle in the animal or human subject.

17. A method of synthesizing an encapsulated high ionic concentration saline nanodroplet comprising the steps:
(a) mixing an activated perfluoroether with poly(propylene glycol)-block-poly(ethylene glycol)-block-poly(propylene glycol)bis(2-aminopropyl ether;
(b) synthesizing a first emulsion by sonicating a mixture of the product of step (a), a perfluoroalkane, and an aqueous salt solution to generate a first emulsion; and
(c) generating a double emulsion by sonicating a mixture of the product of step (c) and a non-ionic surfactant, thereby forming a population of encapsulated saline nanodrops.

18. The method of claim 17, wherein the non-ionic surfactant is Pluronic F-68.

19. The method of claim 17, further comprising the step of functionalizing the encapsulated saline nanodroplets with a reactive group.

20. The method of claim 19, wherein the reactive group is a reactive thiol group.

21. The method of claim 20, wherein the reactive thiol group is 1,2-distearoyl-sn-glycero-3-phosphoethanolamine-N-(polyethylene glycol)-thiol (DSPE-PEG₂₀₀₀-SH).

22. The method of claim 17, further comprising at least one functional moiety conjugated to the reactive thiol group, wherein the functional moiety is a detectable label, a ligand having selective affinity for a cell or a biomarker of a cell, an immunoglobulin or fragment thereof having selective affinity for a cell or a biomarker of a cell, a protecting molecule that reduces immunogenicity of the nanodroplet, or a pharmaceutically active agent.

23. The method of claim 22, wherein the detectable label is a fluorescent dye.

24. The method of claim 22, wherein the at least one functional moiety is conjugated to the reactive thiol group by a linker.

25. The method of claim 24, wherein the linker is sulfosuccinimidyl 4-(N-maleimidomethyl) cyclohexane-1-carboxylate (Sulfo-SMCC).

26. The method of claim 23, wherein the fluorescent dye is indocyanine green (ICG).

27. The method of claim 17, wherein the ligand having selective affinity for a cell or a biomarker of a cell specifically binds to a cell receptor.
